(19) United States
(12) Patent Application Publication
(10) Pub. No.: US 2013/0095078 A1
(43) Pub. Date: Apr. 18, 2013

(54) METHODS FOR REGENERATING SKELETAL MUSCLE

(71) Applicants: Christopher Malcuit, Hudson, OH (US); Tanja Dominko, Southbridge, MA (US); Raymond Lynn Page, Southbridge, MA (US); George D. Pins, Holden, MA (US); Jennifer Makridakis, Mendon, MA (US)

(72) Inventors: Christopher Malcuit, Hudson, OH (US); Tanja Dominko, Southbridge, MA (US); Raymond Lynn Page, Southbridge, MA (US); George D. Pins, Holden, MA (US); Jennifer Makridakis, Mendon, MA (US)

(21) Appl. No.: 13/634,075

(22) PCT Filed: Mar. 11, 2011
(86) PCT No.: PCT/US2011/028169

§ 371 Date: Dec. 17, 2012

Related U.S. Application Data
(60) Provisional application No. 61/312,705, filed on Mar. 11, 2010.

(51) Int. Cl. A61K 38/39; A61M 37/00; A61K 35/12; A61K 35/34; A61K 35/44

(52) U.S. Cl. CPC A61K 38/39; A61K 35/34; A61K 35/44; A61K 35/33; A61M 37/00
USPC 424/93.7; 514/17.2; 604/93.01

(57) ABSTRACT

An engineered muscle construct in the form of a braided collagen microthread scaffold is provided. The microthread scaffold can be used with or without cells as engineered skeletal muscle. The microthread scaffold can also be used to promote cell attachment and growth to deliver cells to a large muscle defect to stimulate muscle regeneration. Methods for making a muscle construct, seeding cells onto microthread scaffolds and treating muscle defects are also provided.

### STATEMENT REGARDING FEDERALLY SPONSORED RESEARCH OR DEVELOPMENT

This invention was made, in part, with Government support under grant number EB-005645 awarded by the National Institutes of Health and contract # W911NF09C0004 awarded by DARPA and funded by the Army Research Office. The Government has certain rights in the invention.

### BACKGROUND OF THE INVENTION

Injury to skeletal muscle tissue induced by intense exercise or minor trauma is repairable, to a point, by the activation of a small pool of quiescent muscle progenitors, termed ‘satellite’ cells, residing beneath the basal lamina of myofibers, which are capable of self-renewal, differentiation into proliferating myoblasts, and terminal differentiation by fusion into functional myotubes. In the event of large-scale soft tissue traumas, such as those seen during combat-associated injury, extensive loss of full-thickness native tissue architecture renders the wound site unable to support regeneration by this mechanism. Rather, following an acute inflammatory response after this type of injury, a provisional fibrin matrix is established from trauma-associated blood clotting, and this matrix is then infiltrated by type-I collagen-producing fibroblasts, resulting in the formation of scar tissue. Although scar formation limits overall loss of tissue and serves as a scaffold for wound remodeling, it precludes the re-establishment of functional skeletal muscle, nervous, and vascular tissue components. While autologous myogenic cellular replacement therapies have shown promise in animal models and may hold promise for clinical models of minor traumas, success with this approach is constrained by the limited scalability of donor cells to required therapeutically-relevant doses as well as the lack of appropriately organized scaffolding within the wound bed, capable of supporting myotube formation and alignment, vascularization, and re-innervation.

Developing a scaffold to address full-thickness replacement of skeletal muscle must address several key criteria. The material must be biocompatible, capable of three-dimensional organization, promote native tissue ingrowth and alignment, and be biodegradable yet mechanically stable. In vitro and in vivo studies using synthetic materials and natural polymers, including polyglycolic acid (PGA), poly(s-caprolactone) (PCL), hyaluronic acid, alginate, fibrin, and acellular matrices, have been shown to support myoblast migration, differentiation, fusion, and in some cases, revascularization. Fibrin gels have been shown to increase healing outcomes for a variety of wound types including skeletal muscle, have been used to stimulate and study muscle differentiation in vitro, and have been employed as a delivery vehicle for myogenic cells in vivo. However, most in vivo studies have been restricted to assessment of engraftment potential of different cell types or the ability of specific extracellular materials in either non-injured muscle or in injuries (e.g. cryo, ischemia, cardiotoxin) where the extracellular scaffold of tissue is left intact.

### BRIEF SUMMARY OF THE INVENTION

In one embodiment, the methods of these teachings are directed to binding growth factors to the surface of crosslinked braided collagen scaffolds to promote muscle-derived fibroblastic cell (MDFC) attachment and growth, which serves as a platform for delivering cells to large muscle defects for muscle regeneration. The surface and mechanical strength of the braided collagen scaffold was characterized to verify that the scaffolds are suitable for integrating into native skeletal muscle. In addition, quantitative and qualitative analysis of cell attachment, growth, and alignment through immunocytochemistry and cell growth assays confirmed that surface modifications facilitate the growth of MDFCs on braided collagen scaffolds.

In another embodiment, the methods of these teachings are directed to making an engineered muscle construct from self-assembled collagen microthreads, as well as the development of a method to seed MDFCs onto a three dimensional scaffold. In another aspect, the methods of these teachings are directed to the procedures used to characterize the braided collagen scaffold and the cell attachment and growth both quantitatively and qualitatively.

In another embodiment, the methods of these teachings are directed to using a scaffold system composed of fibrin microthreads as an efficient delivery system for cell-based therapies and for improving regeneration of a large defect in muscle. The tibialis anterior (TA) of the mouse was used as an in vivo model. In one aspect, implanting cell-loaded fibrin microthread bundles implanted into a skeletal muscle resection reduced the overall fibroplasia-associated deposition of collagen in the wound bed and promoted in-growth of new muscle tissue. When fibrin microthreads were seeded with stem-like human cells, implanted cells contributed to the nascent host tissue architecture by forming skeletal muscle fibers, connective tissue, and PAX7 positive cells. Stable engraftment was observed at 10 weeks post-implant and was accompanied by reduced levels of collagen deposition.

The microthreads of these teachings may be referred to as compositions as a whole or to one or more of their component parts as a medical device because their physical configuration and features allows them to be administered and subsequently confer a benefit on a patient who has a damaged skeletal muscle (e.g. muscle tissue injured by trauma, a disease, or disorder).

More specifically, the compositions can include a polymer configured as a thread or a plurality of threads (which may be bundled as described below), each having a leading end and a trailing end. The present compositions can also include a plurality of biological cells and/or one or more therapeutic agents.

Many different types of polymers and many combinations of polymers are useful (i.e., the threads within a bundle may be, but are not necessarily, composed of the same types of polymers). For example, the polymer configured as a plurality of threads can include a naturally occurring polymer such as a proteoglycan, a polypeptide or glycoprotein, or a carbohydrate or polysaccharide. More specifically, the proteoglycan can be heparin sulfate, chondroitin sulfate, or keratin sulfate; the polypeptide or glycoprotein can be collagen, fibrin, fibronectin, firbrinogen, elastin, tropoelastin, gelatin, silk; and the carbohydrate or polysaccharide can be hyaluronan, a starch, alginate, pectin, cellulose, chitin, or chitosan.

The microthreads can be “free” or can be braided, bundled, tied, or otherwise collected to form filaments. The microthreads can have a diameter of about 0.2 to 1,000 μm (e.g., about 2-100; 10-100; 20-100; 50-100; 60-100; 100-500; or 500-1,000 μm, inclusive) and, when bundled can include about 3-300 microthreads (e.g. about 4, 10, 15, 25, 50, 100, 200 or 300 microthreads).

The microthread surfaces can be treated or modified. For example, braided, bundled, tied, or collections of threads can be crosslinked. Chemical crosslinking agents such as 1-ethyl-3-(3-dimethyl aminopropyl) carbodiimide (EDC) and N-hydroxysuccinimide (NHS). In addition, crosslinking can be in the presence of other surface modifying agents, for example heparin can be crosslinked to the threads, braids, or bundles.

The cells can vary but will be cells that facilitate repair of the damaged muscle tissue, whether through their own differentiation, integration and/or function or by promoting the survival, differentiation, integration and/or function of cells within the patient's tissues (or both). Thus, the cells associated with the microthreads can be, or can include, differentiated cells such as muscle-derived cells, muscle-derived fibroblastic cells, skeletal muscle satellite cells, satellite like cells, primary skeletal muscle cells, fibroblasts, and endothelial cells. The cells can also be stem cells, precursor cells, or progenitor cells (i.e., any cells that are not fully or terminally differentiated including dedifferentiated cells), such as dedifferentiated fibroblast cells, stem-like cells, myoblasts, induced pluripotent stem cells (iPS cells), muscle progenitor cells, embryonic stem cells, and mesenchymal stem cells. The source of the cells can also vary. For example, the cells may be, or may include, those obtained from the same patient who is subsequently treated with the composition (i.e., the cells can be autologous) or they may be obtained from another person (i.e., the cells can be allogeneic).

Where a therapeutic agent is included, it may be any type of agent that facilitates repair of patient's tissue, either directly or indirectly, or confers some other benefit on the patient. For example, the therapeutic agent can be a protein-based agent such as a polypeptide growth factor or an antibody; a vitamin or a mineral; an antimicrobial agent (e.g., an anti-viral, anti-fungal, or antibiotic), or a small organic molecule. The therapeutic agent can affect the cells within the present compositions and/or the cells within the patient's own tissues. Suitable growth factors include an FGF (e.g., FGF-2), VEGF, an IGF (e.g, IGF-I), a PDGF, and EGF, an NGF, a BDNF, or a metalloprotease.

One embodiment of these teachings features methods of making cell-containing compositions that can be used to deliver cells to a patient. To make those compositions, the microthreads described herein can be placed in a cell culture vessel with cells such that the cells become associated with the plurality of threads to form the cell-containing compositions. The precise nature of the association can vary. The cells can associate with the microthreads just as they would with any other biocompatible or inert substrate.

The teachings further encompass methods of making a muscle repair composition comprising the microthreads described herein. These method can include the steps of: providing or introducing cells that induce or enhance the repair or regeneration of muscle tissue into a culture medium comprising a polymer thread (or a plurality of threads configured as a bundle or braided) having a leading end and a trailing end; culturing the cells under conditions that allow the cells to associate with the thread; and removing the thread and associated cells from the culture medium. Alternatively, or in addition to the cells, the microthreads can be used to deliver a therapeutic agent, in which case the methods of making the muscle repair composition will include a step of associating a therapeutic agent with a polymer thread (or a plurality of threads configured as a bundle or braided).

Methods of treatment are also feature of the present teachings. For example, a patient who has a skeletal muscle defect can be treated by administering compositions described herein to the site of the defect. The microthreads that can be so administered include microthreads with or without associated cells.

Other features of the present teachings will be described below and are illustrated in the accompanied drawings.

### BRIEF DESCRIPTION OF THE DRAWINGS

FIG. 1 is a diagram of braided collagen scaffold preparation;

FIG. 2 is phase images of single threads, six thread braids, and 18 thread braids;

FIGS. 3A-D are photographs showing the immunocytochemical verification of the presence of FGF-2 on collagen threads;

FIGS. 4A-C are bright field images showing braided collagen thread cross-sections;

FIGS. 5A-B are graphs showing load-elongation curves;

FIG. 6 is a graph showing characteristic load-elongation relationship for braided collagen microthreads;

FIGS. 7A-B are photographs showing cross-sections of braided collagen threads;

FIG. 8 is a graph showing characteristic stress-strain curve relationship for braided collagen microthreads;

FIGS. 9A-B are bar graphs showing ultimate load and UTS at failure for braided collagen microthreads;

FIGS. 10A-B are bar graphs showing strain at failure and maximum tangent modulus for braided collagen microthreads;

FIG. 11 is a diagram of the preliminary seeding method;

FIGS. 12A-C are photographs showing MDFCs labeled with Mitotracker Green on braided collagen scaffolds;

FIG. 13 is a diagram of the seeding of MDFCs onto a braided collagen scaffold;

FIG. 14 is photographs of Hoechst stained MDFCs seeded onto braided collagen scaffolds;

FIG. 15 is a bar graph comparing MDFC attachment seeding with different channel dimensions;

FIG. 16 is a bar graph comparing total MDFC attachment seeding with different channel dimensions;

FIG. 17 is a bar graph showing the percentage of MDFCs seeded that attached to the braided collagen scaffold;

FIG. 18 is a diagram of MDFCs on a braided collagen scaffold for cell distribution analysis;

FIGS. 19A-F are photographs of Hoechst stained MDFCs on braided collagen scaffolds on day 1;

FIG. 20 is a bar graph showing MDFC attachment for different surface modifications;

FIG. 21 is a graph showing cell distribution on braided collagen scaffolds after 1 day;

FIG. 22 is photographs of Hoechst stained images of MDFC growth on braided collagen scaffolds;

FIG. 23 is a bar graph comparing cell growth with different surface modifications;

FIG. 24 is a bar graph comparing cell growth after 7 days in culture;

FIGS. 25A-F are graphs showing cell distribution for braided collagen scaffolds over 7 days;

FIG. 26 is a bar graph showing MDFC total attachment for different surface modifications;

FIG. 27 is a bar graph showing percentage of MDFC seeded that attached to the braided collagen scaffolds;

FIG. 28 is a bar graph showing total cell growth after 7 days in culture;

FIG. 29 is a bar graph showing the effect of surface modifications on growth rate;

FIG. 30 is photographs showing H&E stained braided collagen threads at 1 and 7 days;

FIG. 31 is photographs showing qualitative analysis of cell density of Hoechst stained MDFCs on braided collagen scaffolds with different surface modifications;

FIG. 32 is photographs showing analysis of alignment using phalloidin staining of braided collagen scaffolds with different surface modifications;

FIGS. 33A-D are photographs of RT-PCR analysis of transcripts associated with pluripotency (A) and myogenesis (B) in adult human muscle-derived cells and photographs of immunocytochemistry analysis (ICC) of pluripotency-associated proteins in primary human muscle-derived cells cultured in ELS (C) and immunocytochemistry analysis of myogenic proteins in muscle derived cells cultured in ELS or Standard culture conditions (D);

FIGS. 34A-C are photographs of Fibrin microthread before cell seeding (A), after seeding with muscle derived fibroblasts (B) and phase contract merged image (C);

FIGS. 35A-C are photographs showing morphology of skeletal muscle wounds at 2 days, 1 week, 2 weeks and 10 weeks after implantation of a cell-populated, fibrin microthread implant. (A), gross muscle tissue structure following microthread loaded cell implants at indicated time points. (B), and high magnification of Trichrome/eosin stained sections of wound area marked by the presence of fibrin microthreads implanted with embedded carbon particles (C);

FIGS. 36A-C are photographs of representative histological sections of wound healing in untreated wounds with no implant (A) and microthread implanted wounds (B) and a bar graph showing quantitation of collagen (C);

FIGS. 37A-E are photographs showing immunohistochemistry of implanted wounds with mouse anti-human nuclear antigen antibody 2 days (A) and 2 weeks post-implant (B), negative control (C), positive control (D) and genomic PCR for human and mouse-specific DNA sequences (E); and

FIGS. 38A-D are photographs showing immunohistochemistry of implanted wounds with anti-PAX 7 antibody, human muscle positive control (A), mouse muscle negative control (B), and fibrin microthread/human cell implants (C) at 40× and showing frequency of PAX7 positive cells in control human muscle and in fibrin microthread/human cell implanted wounds (D).

### DETAILED DESCRIPTION OF THE INVENTION

Braided collagen microthreads were seeded with muscle-derived fibroblastic cells (MDFCs) as a scaffold to aid in muscle regeneration by providing a structure to create longitudinally aligned myotubes. Although braided collagen microthreads are not parallel to one another like each myofiber in native skeletal muscle, by weaving the microthreads together, the scaffold structure can be maintained without thread spreading from one another during hydration. When full thickness defect occurs, the entire depth of the muscle is damage, resulting in the destruction of many myofibers. Since the diameter of one microthread is smaller than that of a myofiber, which ranges from 20 to 100 μm, by braiding the threads together, the dimensions of the microthreads can be increased to fill a larger defect area. Studies have shown using a biomaterial with stiffness close to that of native muscle, 12 kPa, for engineering skeletal muscle can affect the length, alignment, and contractibility of the formed myofibers. Using EDC/NHS crosslinking, the mechanical properties of the braided collagen microthreads can be controlled to enhance myofiber formation. When a large muscle defect occurs, the native skeletal muscle cannot regenerate itself since the satellite cells within the basal lamina have been destroyed, so there is a need for a way to deliver satellite like cells to the defect area to enhance regeneration. Attaching heparin and FGF-2 to the surface of the braided collagen scaffold serves to help bind, modulate, and release FGF-2 to the seeded cells and the surrounding environment. In addition, the presence of FGF-2 on the surface of the scaffold provides a method to release a controlled amount of the growth factor to the cells to maintain the undifferentiated state of muscle derived fibroblast cells. This will ensure a population of dedifferentiated fibroblast cells will be delivered to the defect site to behave like satellite cells to induce muscle regeneration.

### Characterization of the Structural Properties of Braided Collagen Scaffolds

To test whether braided collagen scaffolds are suitable for integrating into a skeletal muscle defect and maintaining mechanical stability, three braids of six individual self assembled collagen microthreads were braided together in a three strand braid to form a final eighteen microthread braided collagen scaffold. Next, the scaffolds were crosslinked using EDC/NHS with or without heparin and FGF-2 in concentrations of 5 ng/mL, 10 ng/mL, or 50 ng/mL was passively adsorbed to the surface. The braided collagen scaffolds were characterized through immunocytochemistry and mechanical testing. Due to limitations involved with imaging a three-dimensional braided scaffold, single threads were passively adsorbed with different concentrations of FGF-2 and treated with the standard protocol for immunocytochemistry. This was used to show that different concentrations of FGF-2 were bound to the surface of the braided collagen scaffolds. Next, to test the mechanical stability of the braided collagen scaffolds, uncrosslinked and crosslinked scaffolds were loaded in uniaxial tension to extrapolate the measured ultimate load at failure, strain at failure, and maximum tangent modulus (MTM).

Type I Collagen Extraction from Rat Tendon

Acid-soluble type I collagen was extracted from rat tail tendons as previously described. Briefly, tendons were removed from 13 Sprague-Dawley rat tails with a hemostat, rinsed in phosphate buffered saline (PBS, pH 7.4), and dissolved in 1600 mL of 3% (vol/vol) acetic acid overnight at 4° C. The collagen solution was filtered through layered cheesecloth and centrifuged for 2 hours at 8500 rpm at 4° C. Discarding the pellet, a salt precipitation was performed where 320 mL of 30% NaCl (wt/vol) solution was dripped into the supernatant. The solution was allowed to sit overnight at 4° C. The entire solution was then centrifuged at 4° C. for 40 minutes at 4900 rpm, and the resulting pellet was resuspended on a stir plate in 400 mL of 0.6% (vol/vol) acetic acid at 4° C. until the pellet had dissolved completely. The solution was placed in dialysis membranes (Spectrum Laboratories, Inc., Rancho Dominguez, Calif.) and dialyzed at room temperature in 1 mM HCl changing the dialysate every 4 hours until the solution was clear. The type I collagen solution was lyophilized and stored at 4° C. Prior to use, the lyophilized collagen fleece is dissolved in 5 mM HCl at a concentration of 10 mg/mL.

### Self Assembled Collagen Thread Extrusion

Self-assembled collagen threads were produced from acid soluble type I collagen using methods described previously. Briefly, type I collagen (10 mg/mL in 5 mM HCl) was placed in a 5 mL syringe connected to a polyethylene tube with an inner diameter of 0.86 mm (Becton Dickinson, Franklin, N.J.). Using a syringe pump, the solution was extruded through the tubing at a rate of 0.255 mm/min into a 37° C. bath of fiber formation buffer (pH 7.4, 135 mM NaCl, 30 mM Tris Base, 30 mM Tris HCl, and 5 mM NaPO₄ dibasic; Sigma, St. Louis, Mo.) and incubated for 24 hours. The formed threads were transferred to a 37° C. bath consisting of fiber incubation buffer (pH 7.4, 135 mM NaCl, 10 mM Tris Base, 10 mM Tris HCl, and 30 mM NaPO₄ dibasic; Sigma) for an additional 24 hours. The threads were then washed in a 37° C. bath of distilled water for 24 hours to remove the salt, air dried, and stored at room temperature in a dessicator until use.

### Braided Scaffold Preparation

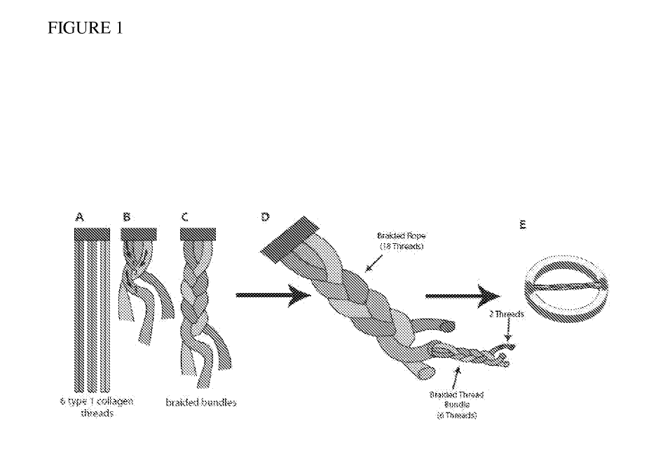

To create braided collagen scaffolds, first six type I collagen microthreads were attached to a single point and split into three groups of two threads each (FIG. 1A). The grouped threads were braided together with 28 crossovers per centimeter. A crossover is defined as one group crossing over an adjacent group (FIGS. 1B and C). The final braided scaffold was produced by attaching three six-thread braids to a single point and braiding them together with 26 crossovers per centimeter (FIG. 1D). The final braided scaffolds containing 18 self-assembled threads were attached to PDMS (Dow Corning, Midland, Mich.) rings with an inner diameter of 14 mm using Silastic Silicone Medical Adhesive Type A (Dow Corning, Midland, Mich.) in order to easy fit inside a 12 well tissue culture plate (FIG. 1E).

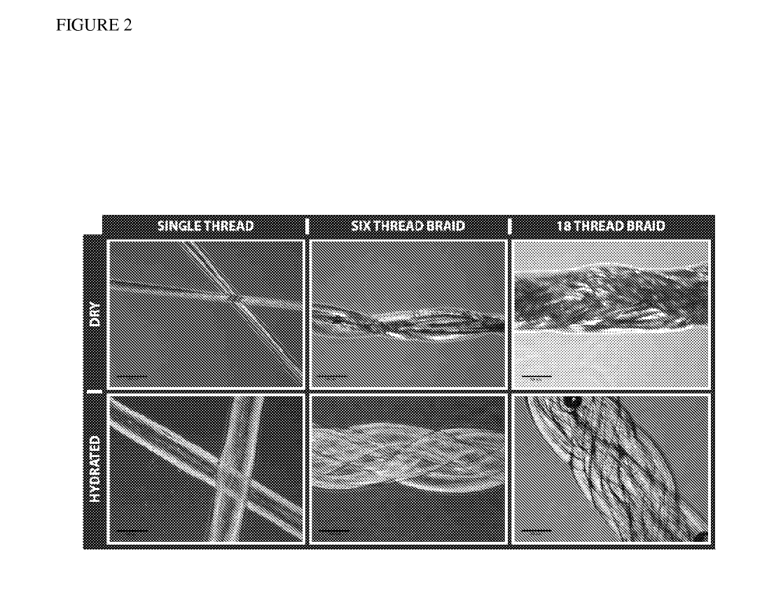

The phase images in FIG. 2 and Table 1 below compare the size of single threads, 6 thread braids, and 18 thread braids both dry and hydrated in PBS. Phase images were obtained using an Olympus IX81 motorized inverted microscope coupled to a 12-bit Hamamatzu CCD camera and processed using Slidebook®.

TABLE 1
Width comparison of single threads, 6 thread braids, and 18 thread braids

| Thread | Width Dry | Width Hydrated |
|---|---|---|
| Configuration | (μm ± S.D.) | (μm ± S.D.) |
| Single | 50 ± 20 | 180 ± 20 |
| 6 Braid | 250 ± 30 | 400 ± 50 |
| 18 Braid | 450 ± 25 | 600 ± 50 |

Microthread Crosslinking with Heparin

Braided collagen scaffolds were crosslinked using the chemical crosslinker 1-ethyl-3-(3-dimethyl aminopropyl) carbodiimide (EDC; Sigma) and N-hydroxysuccinimide (NHS; Sigma) with and without heparin sodium salt (Calbiochem, Gibbstown, N.J.). In a sterile field, braided collagen scaffolds were inverted and inserted into a 12 well plate with one scaffold per well, and they were washed with 70% (vol/vol) ethanol 4 times for 30 minutes each and 40% (vol/vol) ethanol 5 times for 15 minutes each to sterilize. Subsequently, the scaffolds were submerged in 3 mL of sterile filtered 40% (vol/vol) ethanol including 50 mM 2-morpholinoethane sulphonic acid (MES, pH 5.0; Sigma) for 30 minutes at room temperature. Next, the scaffolds were incubated in 2 mL of sterile filtered 40% (vol/vol) ethanol including 50 mM MES, 14 mM EDC, 8 mM NHS, with and without 100 μg/mL heparin for 4 hours at room temperature. The scaffolds were washed in 70% (vol/vol) ethanol 5 times for 10 minutes each with a final overnight wash at 4° C.

FGF-2 Binding through Passive Adsorption

Fibroblast growth factor (FGF-2; Chemicon, Temecula, Calif.) in varying concentrations was passively adsorbed to the surface of braided collagen scaffolds and crosslinked with EDC/NHS and heparin using methods previously described. Briefly, scaffolds were washed 5 times for 10 minutes with sterile Dulbecco's phosphate buffered saline (DPBS, pH 7.4) without calcium and magnesium at room temperature. Subsequently, the chamber walls, PDMS ring, silicone adhesive and nonspecific binding sites on the braided collagen scaffolds were blocked using a blocking solution of 3 mL of sterile filtered DPBS containing 0.25% (wt/vol) bovine serum albumin (BSA; Sigma) for 1 hour at room temperature. Next the blocking solution was aspirated from each well, and replaced with 2 mL of sterile DPBS containing 0.25% (wt/vol) BSA with FGF-2 at a concentration of either 5 ng/mL, 10 ng/mL, or 50 ng/mL. The scaffolds were incubated for 2 hours at room temperature. The braided collagen scaffolds were washed in DPBS 5 times for 10 minutes each and stored at 4° C. in DPBS until use.

### Braided Collagen Scaffold Structural Characterization Characterization of Bound FGF-2

To verify that FGF-2 bound to the surface of the scaffolds immunocytochemistry was performed. Due to imaging limitations of the geometry of braided collagen scaffolds, single collagen threads were used to characterize the localization of FGF-2 on the surfaces. Single collagen threads were EDC/NHS crosslinked in the presence of heparin and then loaded with FGF-2 at the concentrations mentioned above. Single collagen threads that were EDC/NHS crosslinked in the presence of heparin were used as negative controls. DPBS was removed from the wells by aspiration, and the threads were incubated at room temperature in 300 μL of 1 μg/mL FGF-2 goat polyclonal IgG (Santa Cruz Biotechnology, Inc., Santa Cruz, Calif.) in PBS with 0.05% Tween-20 (Promega Corporation, Madison, Wis.) for 30 minutes. The threads were washed with 5004 of PBS with 0.05% Tween-20 for 5 minutes three times. The threads were then incubated in 300 μL of 5 μg/mL Alexa Fluor 647 donkey anti-goat IgG (Invitrogen, Carlsbad, Calif.) in PBS with 0.05% Tween-20 for 30 minutes. They were then washed in 500 μL of PBS with 0.05% Tween-20 for 5 minutes twice. Single collagen threads were imaged using fluorescence microscopy on an Olympus IX81 motorized inverted microscope coupled to a 12-bit Hamamatzu CCD camera and processed using Slidebook®.

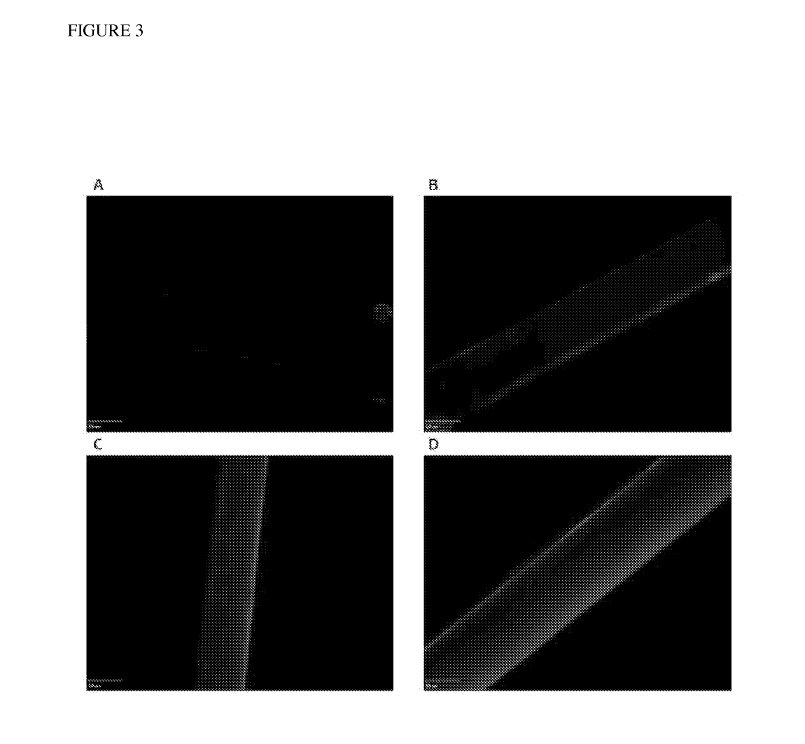

To verify and characterize the localization of FGF-2 on braided collagen scaffolds, different concentrations of FGF-2 were bound to single collagen threads that were crosslinked with EDC/NHS in the presence of heparin. The threads were immunostained and the results are shown in FIG. 3. Threads crosslinked with EDC/NHS and heparin in the presence of FGF-2 showed FGF-2 on the surfaces of the threads when compared to the control braids that were crosslinked with EDC/NHS and heparin, but not exposed to FGF-2 (FIG. 3A). Immunocytochemical analysis of threads exposed to 5 ng/mL FGF-2 showed FGF-2 having inconsistent coverage on the surface (FIG. 3B). To determine if this inconsistency was due to an imaging artifact, it would be beneficial to image the threads using a scanning confocal microscope. Although it is apparent that FGF-2 was present on the surface, it is not uniform along the length of the thread. Threads exposed to 10 ng/mL FGF-2 (FIG. 3C) and 50 ng/mL FGF-2 (FIG. 3D) showed similar localization and uniform coverage of FGF-2 along the length of the thread. Threads exposed to 50 ng/mL FGF-2 seem to have a more FGF-2 bound to the surface due to the higher fluorescence intensity across the whole surface. However, these differences were not evaluated quantitatively.

The immunocytochemistry of the single collagen threads verified that FGF-2 was bound to the surface, and the fluorescence expression increased depending on the concentration of FGF-2 in the solution. Initially, immunocytochemistry performed on braided collagen scaffolds to verify the localization of FGF-2 on the surface did not give conclusive results due to the limitations of imaging the entire braid in one plane of the z-axis (results not shown). Since the surface area of single collagen microthreads exposed to EDC/NHS with heparin and FGF-2 is much smaller than braided collagen threads (5.46±2.55 mm² to 19.05±3.90 mm² respectively), binding heparin and FGF-2 to single thread using the same protocol will validate that braided threads have it on the surface. Threads exposed to 5 ng/mL FGF-2 showed minimal surface binding with areas containing higher fluorescence intensity than others, which suggests that the heparin FGF-2 binding was not homogeneous throughout the surface. When threads were exposed to 10 ng/mL and 50 ng/mL of FGF-2, the difference between the fluorescence intensity along the thread was less apparent, however the 50 ng/mL FGF-2 threads showed a greater overall surface coverage.

Studies show that culturing fibroblasts in culture medium supplemented with 4 ng/mL FGF-2 in low oxygen conditions (5% O₂, 5% CO₂) for an weeks allows for dedifferentiation into stem-like cells, and with continued exposure to FGF-2, the stem cell marker expression could be maintained. Levenstein et al. showed that culturing human embryonic stem cells with media supplemented with 100 ng/mL FGF-2 maintained the pluripotency of the cells through multiple passages in the absence of fibroblasts, which is standard for growing stem cells. Mizuno et al. was able to induce differentiation of iPS cells reprogrammed from fibroblasts into myoblasts and maintain the sternness for 24 weeks on Matrigel-coated plates. By binding FGF-2 to the surface of the braided scaffolds, the cells will have prolonged exposure to the growth factor during in vitro culture and after in vivo implantation. Since FGF-2 has a short half-life, approximately 12 hours in vitro, when not electrostatically bound to a surface, binding heparin to the scaffold using EDC/NHS before passive adsorption of FGF-2, helps to maintain the stability of the growth factor to ensure extended cell exposure.

Previous studies indicate that immobilizing heparin onto the surface of insoluble collagen sponges and films doubles the amount of FGF-2 that adsorbs to the surface compared to sponges without heparin. It is envisioned that braided collagen scaffolds may use larger quantities of heparin and FGF-2. Although it is clear that both are present on the surface of the scaffold, for long term studies, FGF-2 needs to be available during both in vitro culture and in vivo implantation studies. Wissink et al. crosslinked insoluble collagen films with different molar ratios of EDC/NHS to heparin. The results showed that molar ratios between 0.4 and 0.6, which corresponds to 14 mM EDC, 8 mM NHS, with between 1.5 mM and 1.0 mM heparin respectively, yielded 20-30 mg of heparin per gram of collagen immobilized within the sponge. It was found that a maximum of 22% of the FGF-2 added to solution will bind to the heparin on the surface, leading to the conclusion that one FGF-2 molecule will bind to heparin per 1000 heparin molecules. When detecting how much FGF-2 is released from the scaffold over time, the study showed that after 10 days approximately 60% of the growth factor was still present within the sponge. Pieper et al. crosslinked collagen sponges with 960 μM heparin sulfate and 7 μg/mL FGF-2, and found that 36% of the loaded FGF-2 was bound to the scaffold, with 53% of the growth factor being released after 4 weeks.

Therefore, it is envisioned that by crosslinking the braided collagen scaffolds with 1.9% (w/v) heparin and passively adsorbing concentrations of FGF-2 between 50 ng/mL and 100 ng/mL to the surface, the rate of release can be predicted to create the optimal environment for maintained sternness over long intervals. Based on the study by Wissink et al., by adsorbing 50 ng/mL FGF-2 in 1.0 mL of solution, one can predict approximately 11 ng will bind to the surface, which will correlate to releasing 4.5 ng to the cells in the first 10 days of culture. This would be consistent with the concentration of FGF-2 that cells are exposed to when it is incorporated into the culture medium in vitro.

Besides maintaining the cell phenotype, FGF-2 promotes vascularization of a wound site when implanted into a defect. Studies show FGF-2 upregulates vascular endothelial growth factor (VEGF), which stimulates angiogenesis, and prevents the degradation of the capillaries formed. Nillesen et al. EDC/NHS crosslinked collagen sponges with heparin and FGF-2, resulting in 68 mg heparin and 1.6 μg FGF attaching per mg scaffold. After extracting the scaffolds from a rat model 21 days after implantation, the FGF-2 scaffolds stimulated proliferation and differentiation of granulocytes, endothelial cells, fibroblasts in the surrounding tissue, which are responsible for revascularization. Pieper et al. found implanting a crosslinked collagen sponge with 60 mg heparin and 1260 ng FGF per gram of the matrix resulted in capillary formation after 4 weeks. Another study found that EDC NHS crosslinking collagen films with 18.6 mg per gram collagen and 3.4 ng per scaffold elicited a vascular response 3 weeks after implantation. Vascularization is imperative for the formation of healthy skeletal muscle since it provides the oxygen needed to maintain the viability of the differentiating satellite cells, so these results indicate that binding FGF-2 to the surface of the braided collagen scaffolds will help initiate vascularization in large muscle defects.

### Mechanical Testing of Braided Collagen Scaffolds

In order to determine the effect of surface modifications on mechanical strength, EDC/NHS crosslinked and uncrosslinked braided collagen threads were analyzed by mechanically loading the hydrated samples in uniaxial tension. Braided collagen scaffolds that were crosslinked with heparin and exposed to FGF-2 were not tested in this study. Braided collagen threads were cut to a sample length of 30 mm with the last 5 mm of each end bound and sealed using Silastic Silicone Medical Adhesive Type A (Dow Corning, Midland, Mich.). For tensile testing, the samples were secured horizontally with 2711 Series Lever Action Fiber Grips (Instron, Norwood, Mass.) on an E1000 ElectroPuls mechanical testing system (Instron, Norwood, Mass.) with a fixed 50 kN Dynacell dynamic load cell (Instron, Norwood, Mass.). The mechanical testing system and data acquisition were controlled using Bluehill 2 Materials Testing software (Instron, Norwood, Mass.). The samples were secured insuring that the silicone adhesive remained outside of the outer grip boundary. An initial gauge length of 7.0 mm was defined as the distance between the inner grip boundaries, and the braids were loaded to failure at a 50% strain rate (3.5 mm/min).

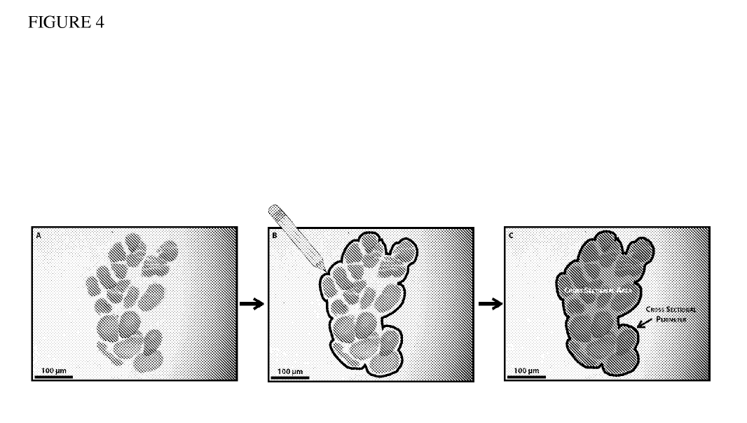

To calculate the ultimate tensile strength, the cross-sectional area of the samples was approximated using histological sections of hematoxylin and eosin stained unseeded braided collagen threads at five different locations. Although the scaffolds shrink due to dehydration after processing, using the histological sections gives a better cross-sectional estimation since using a cylindrical model does not represent the shape accurately. Bright field images were obtained using an Olympus IX81 motorized inverted microscope coupled to a 12-bit Hamamatzu CCD camera and processed using Slidebook®, and analyzed using Image J software (U.S. National Institutes of Health, Bethesda, Md.) (FIG. 4A). The outer edge of the braided collagen threads was traced to measure the cross-sectional area (FIGS. 4B and C). The stress-strain curve, the load at failure, ultimate tensile strength (UTS), stain at failure (SAF), and maximum tangent modulus or stiffness (MTM) were calculated from the data obtained during testing.

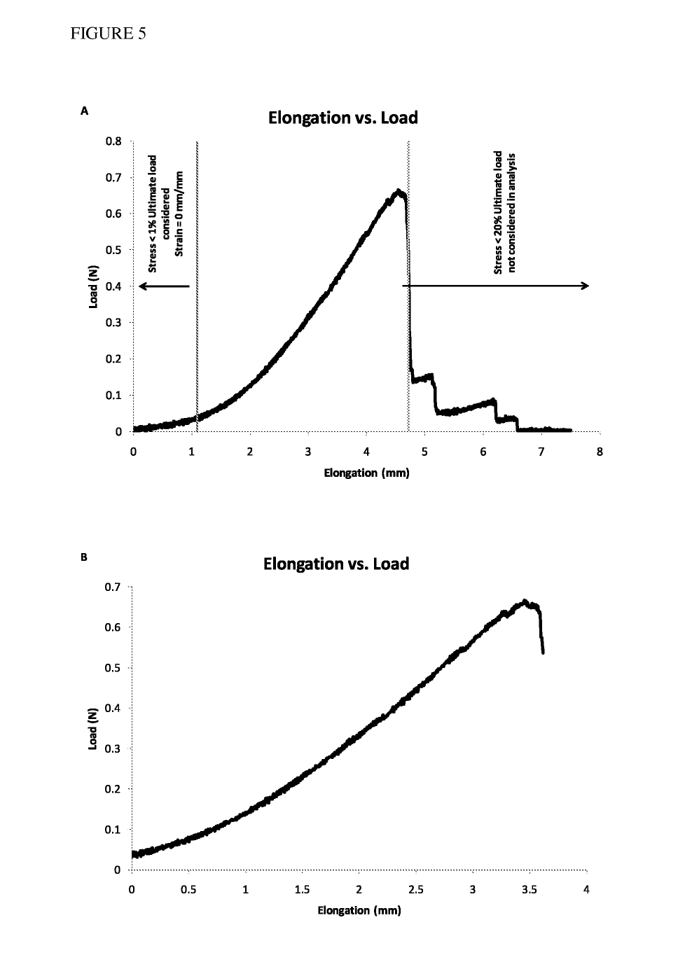

In post processing of the data, a strain of zero was defined as the point where the braided collagen scaffolds were minimally loaded to a threshold of 0.01 grams, or less than 1% the ultimate load of the weakest uncrosslinked scaffold. In addition, load-elongation curves were truncated when the load fell by 20% of the ultimate load, or the point of the initial break (FIG. 5A). After this point, as each individual thread within the braided collagen scaffold broke, they created peaks lower than the ultimate load until each thread in the scaffold failed. For the purpose of this analysis, only the ultimate load was considered (FIG. 5B). The stiffness was defined as the maximum value for a tangent to the stress-strain curve over an incremental strain of 0.03.

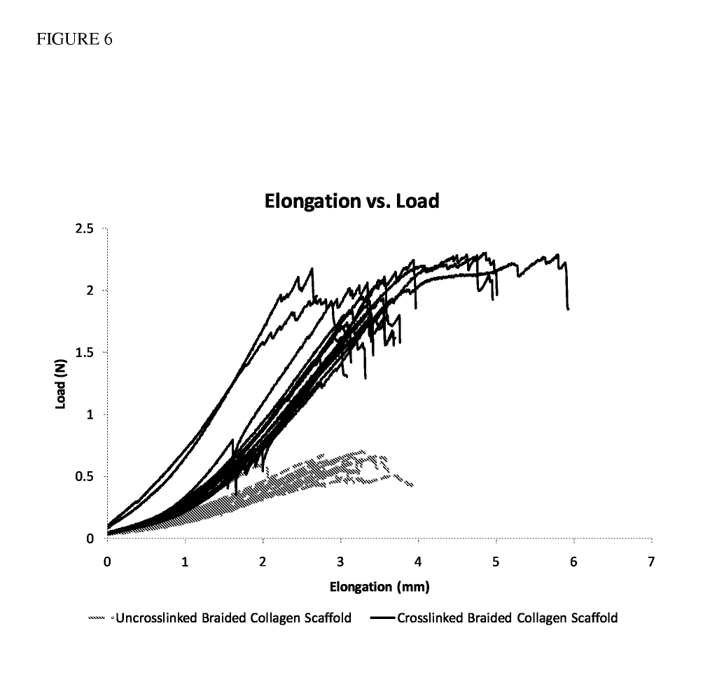

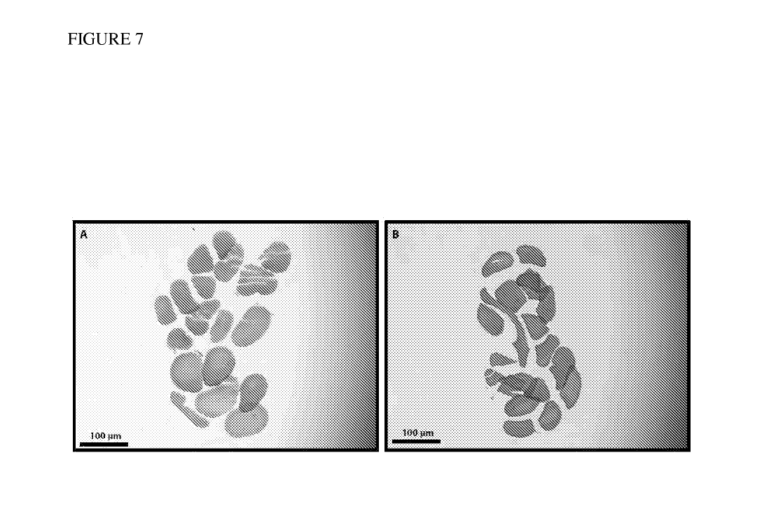

To characterize the mechanical properties of braided collagen scaffolds, uncrosslinked and EDC/NHS crosslinked braids were loaded under uniaxial tension until failure. The results of this analysis are summarized in Table 2. Characteristic load-elongation curves for each of the individual uncrosslinked and crosslinked braided collagen scaffolds showed a generally linear shape with the scaffold failure occurring as the first of the three internal braids fails (FIG. 6). After the point of ultimate failure, the load drops in an incremental manner until each of threads has broken. In addition, each individual braid shows similar curves demonstrating that the production of the braided collagen scaffold from self-assembled type I collagen microthread extrusion to braid development is consistent and reproducible.

TABLE 2
Mechanical properties summary table for braided collagen microthreads

| | | Cross-sectional | | | | Maximum Tangent |
|---|---|---|---|---|---|---|
| | Sample | Area | Ultimate Load | UTS | Strain at Failure | Modulus |
| | Size | (mm² ± SD) | (N ± SD) | (MPa ± SD) | (mm/mm ± SD) | (MPa ± SD) |
| Uncrosslinked | 16 | 0.115 ± 0.025* | 0.591 ± 0.076* | 5.130 ± 0.662* | 0.420 ± 0.064* | 13.60 ± 2.668* |
| Crosslinked | 16 | 0.072 ± 0.013 | 1.979 ± 0.237 | 26.97 ± 2.835 | 0.516 ± 0.118 | 68.52 ± 8.242 |

*Indicates statistically significant differences between uncrosslinked and crosslinked braided collagen scaffolds with p < 0.05 using Mann-Whitney Rank Sum Test.

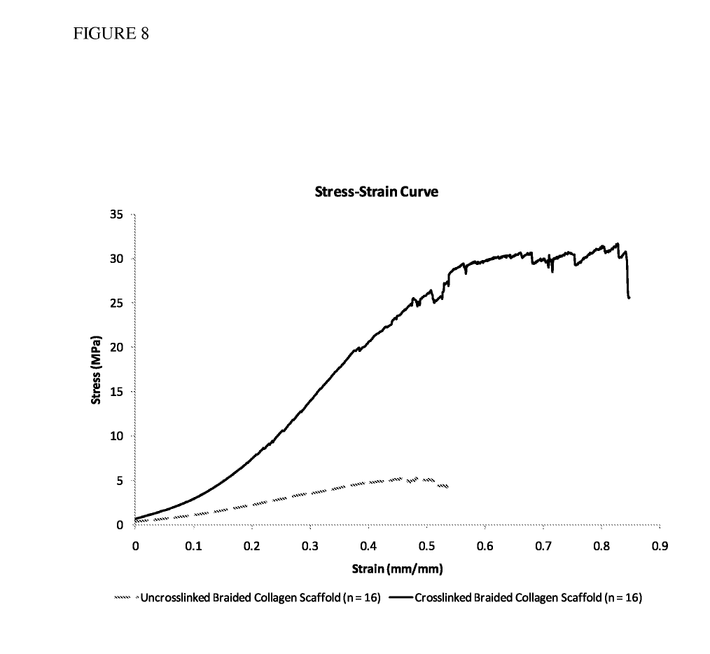

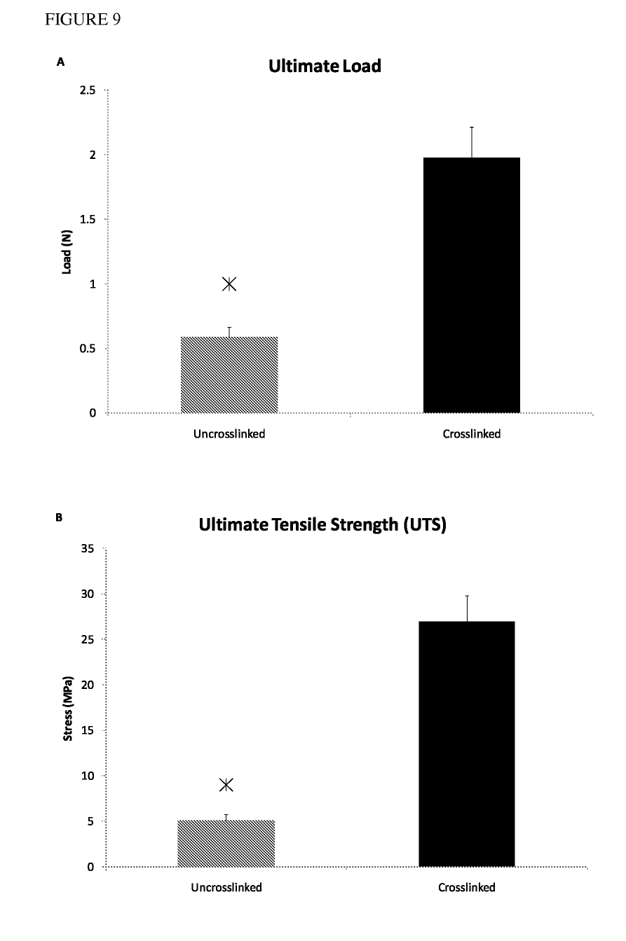

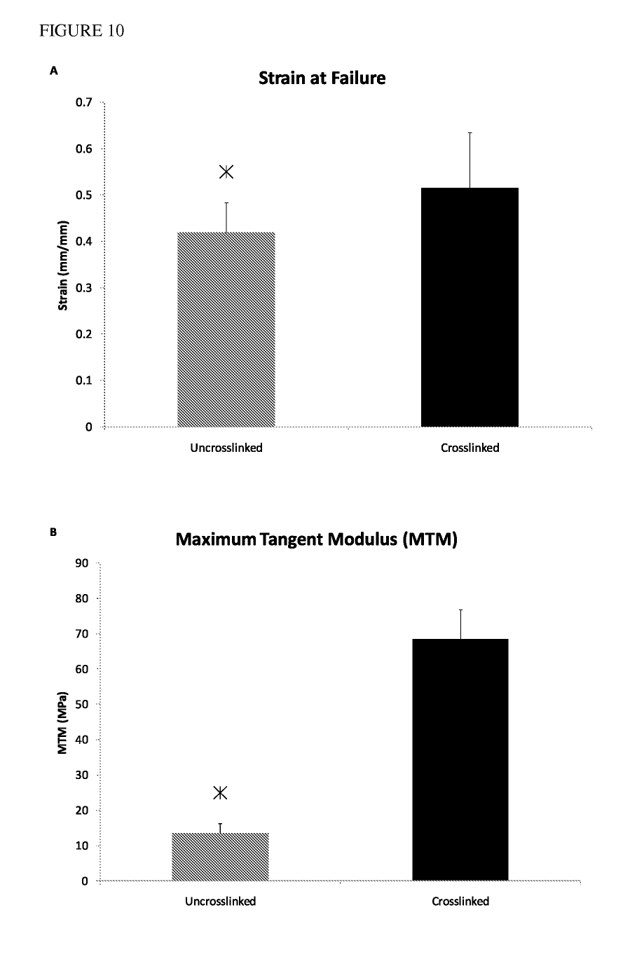

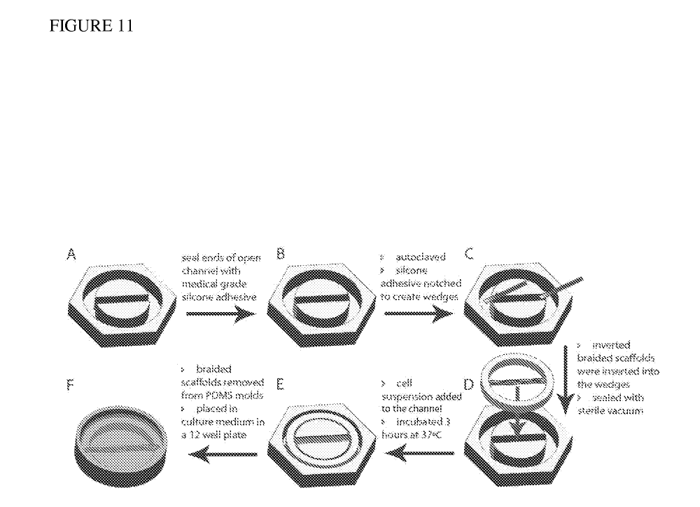

In order to calculate the stress-strain curves of the braids, the cross-section areas were calculated using the histological cross-sections of hydrated braids. The average cross-sectional area of an uncrosslinked and crosslinked braided collagen scaffold was calculated to be 0.115±0.025 mm² (FIG. 7A) and 0.072±0.013 mm² (FIG. 7B) respectively. The representative stress-strain curves comparing uncrosslinked to crosslinked braids also shows they are roughly linear in shape with crosslinked threads withstanding a greater amount of stress per unit strain (FIG. 8). The curve measurements allow for the measurement of the maximum tangent modulus (MTM) of each sample to be calculated as the maximum slope of the stress-strain curve. Relative to uncrosslinked braided collagens scaffolds, the ultimate load and ultimate tensile strength of crosslinked scaffolds were increased significantly by crosslinking using EDC/NHS (FIGS. 9A and B). The crosslinked braids were able withstand an ultimate load almost three times that of uncrosslinked scaffolds. Similarly, the strain at failure and maximum tangent modulus of the crosslinked scaffolds were significantly higher relative to uncrosslinked collagen scaffolds (FIGS. 10A and B). Even though the crosslinked braids are approximately five times stiffer than uncrosslinked scaffolds, the crosslinked braids have a significantly increased strain at failure.

Previous studies by Cornwell et al. on single collagen threads for tissue engineered ligament applications showed crosslinking with EDC increased the mechanical strength significantly compared to uncrosslinked threads. These results suggest EDC crosslinked microthreads have potential to be used for load bearing tissue regeneration. Similarly, tissue engineered skeletal muscle needs to be able to withstand the high tensile loads involved with muscle movement, specifically the muscle contraction involved with myofiber formation and function. A single EDC crosslinked microthread has a cross sectional area larger than a single myofiber when hydrated, approximately 12,100 μm² to 7900 μm², but this would not be sufficient to fill a large muscle defect, which involves severing of multiple myofibers. By braiding 18 collagen threads together to form a scaffold, the cross sectional area is increased to 91,900 μm², which is approximately the size of 12 myofibers. When hydrated, bundles of unbraided threads attached to one another at each end tend to separate from one another, leaving large gaps, which will cause inconsistencies in myofiber formation and little interaction between cells on different threads. By braiding the threads together, the integrity of the structure can be controlled by altering the braiding angle between each thread. This also insures cells on each thread can interact with each other.

Fiber or thread-based scaffolds have been used extensively in ligament regeneration research using collagen, fibrin, silk, PGA, poly-L-lactic acid (PLLA), and polylactic-co-glycolic acid (PLAGA). The mechanical properties of single collagen threads have been researched comparing crosslinked to uncrosslinked conditions, but the strength of braided self-assembled collagen threads previously have not been characterized. For mechanical testing, only uncrosslinked and EDC/NHS crosslinked braided collagen threads were used since modifying the surface with heparin and FGF-2 does not affect the bond between the collagen molecules thus does not affect the strength of the braided structure. Similar to single threads, uniaxial tensile tests of braided collagen scaffolds were conducted and showed a significant increase in strength and stiffness when the braids were crosslinked using EDC/NHS. Due to the large difference in cross sectional area, the results show that braided uncrosslinked threads increases the failure load from 0.389±0.052 N to 0.591±0.076 N. Crosslinking the braids increased the strength of the scaffold at failure to 1.979±0.237 N compared to single crosslinked threads, which has a failure load of 1.33±0.484 N. Interestingly, braiding the collagen threads increased the UTS of the uncrosslinked and crosslinked single threads. The UTS of single uncrosslinked threads was found to be 1.5±0.2 MPa, while braiding the threads increased it to 5.11±0.7 MPa. Crosslinking the single threads using EDC/NHS resulted in a UTS of 11±4 MPa, and braiding these threads increased the UTS to 27±3 MPa. This shows that by braiding the threads to one another, the mechanical strength of the threads can be increased because the woven design prevents catastrophic failure. Interestingly, the strain at failure is the same for uncrosslinked braids and threads, but crosslinking braids resulted in a 3-fold increase in failure strain over crosslinked threads. However, braiding crosslinked collagen threads did not affect the stiffness of the threads as both braids and single threads had an maximum tangent modulus of approximately 68 MPa. Since the threads in braided collagen scaffolds are woven together, it gives the threads added support when pulled uniaxially. A limitation of using braided collagen threads for muscle regeneration pertains to the elasticity of the scaffold since it is significantly higher than that of human muscle, which is 12 kPa. A study varying the stiffness of polyethylene glycol (PEG) hydrogels found that hydrogels with a stiffness of 12 kPa maintained the optimal environment for sustained satellite cell self-renewal and proliferation. It is envisioned that using growth factors, such as FGF-2 or IGF-I, may mimic the environment creating the optimal elasticity to satellite cell self-renewal.

Braided synthetic polymer threads composed of PGA, PLLA, and PLAGA have been researched as possible tissue engineered solutions to repair ligament damage. By keeping the braiding angle constant for each polymer composition, the uniaxial mechanical results showed braided PGA fibers were the strongest with a maximum load of 502 N, and PLLA were the weakest mechanically with a maximum load of 298 N. Synthetic fibers have a much higher mechanical strength compared to collagen braids, which is beneficial for engineered ligaments since they must endure higher loads. For muscle regeneration, such high mechanical strength is not necessary since the loads are not as large. It is envisioned that varying the braiding angle will alter the pore size, or the space between the individual threads, to optimize the ability of cells and nutrients to diffuse into the scaffold for the best tissue ingrowth.

Native skeletal muscle produces isometric forces due to twitch and tetanic contraction, which are impaired when an injury occurs. Iwata et al. characterized the isometric force production of rats with a contusion injury to the plantar flexor muscles, and found the isometric force dropped significantly, approximately 45% that of a healthy skeletal muscle, 2 days after injury onset and returned to normal after 21 days. Another method created large muscle defects in the biceps femoris muscle of a rat model, which showed after 42 days the deficit isometric force remained constant and did not improve. This confirms that the truncated myofibers are unable to regenerate and bridge the gap caused by the defect without a scaffold present leaving the muscle permanently impaired.

The methods of these teachings involve seeding and inducing pluripotency of fibroblasts on the scaffold to initiate muscle regeneration in the wound bed. However, once implanted, the braided collagen scaffolds will have to respond to forces produced from the surrounding myotubes. In addition, the scaffold will have to maintain its structural integrity as the seeded iPS cells differentiate into myoblasts and fuse during tissue remodeling while restoring muscle function. One approach of mimicking this environment in vitro would be to induce differentiation of the iPS cells prior to implantation and measure the contractile forces as well as the force produced by the construct. This can be achieved by electrical and mechanical stimulation as well as measuring the forces produced by stretching and relaxing the construct. Furthermore, since the scaffold will be introduced to native myoblasts and stem cells through endogenous cell migration, it may be beneficial to seed them to the scaffold as well, to evaluate whether it can handle the native tissue response in vitro.

### Developing a Novel Cell Seeding Method to Achieve Uniform and Reproducible MDFC Attachment on Braided Collagen Scaffolds

The first step in developing a novel seeding method to enhance uniformity, efficiency, and reproducibility of MDFC seeding on braided collagen scaffolds was to provide the optimal environment to promote cell attachment onto braided collagen scaffolds. Polydimethylsiloxane (PDMS) molds were created with two posts in the center creating a seeding channel with a dimension of either 2.0 mm by 12 mm or 1.0 mm by 12 mm. Cells were seeded onto braided collagen scaffolds using both channel dimensions, cultured for 24 hours, and analyzed for uniformity to determine which PDMS mold provided the most reproducible results. Second, the most advantageous way to visualize the MDFCs on the braided collagen scaffolds for qualitative analysis was determined. The collagen microthreads exhibit significant autofluorescence when exposed to DNA binding dyes directly, so to address this issue, MDFCs were preloaded with either a MitoTracker Green or Hoechst dye before seeding the cells onto the braided collagen scaffolds. The seeded scaffolds were imaged to determine which dye provided more quantitative information to be used for analysis.

### MDFC Seeding to Braided Collagen Scaffolds

### MDFC Culture and Braided Collagen Scaffold Sterilization

The MDFCs were extracted from the calf flexor muscle of a human adult male through methods described previously. The MDFCs were grown in culture media (40% DMEM, 40% F-12, 20% FC III serum; Mediatech, Inc, Manassas, Va. and Hyclone, Logan, Utah) supplemented with 10 ng/mL epidermal growth factor (EGF, Chemicon, Temecula, Calif.) at ambient conditions (20% O₂ and 5% CO₂) until culture flasks were confluent. Passages 7-8 were used for all cell-seeding experiments. Prior to MDFC seeding, the braided collagen scaffolds were incubated at room temperature with 3% penicillin/streptomycin (Pen/Strep; Gibco BRL, Gaithersburg, Md.) in DPBS (vol/vol) for one week changing the antibiotic solution every 2 days to sterilize scaffolds.

### Preliminary Cell Seeding Method

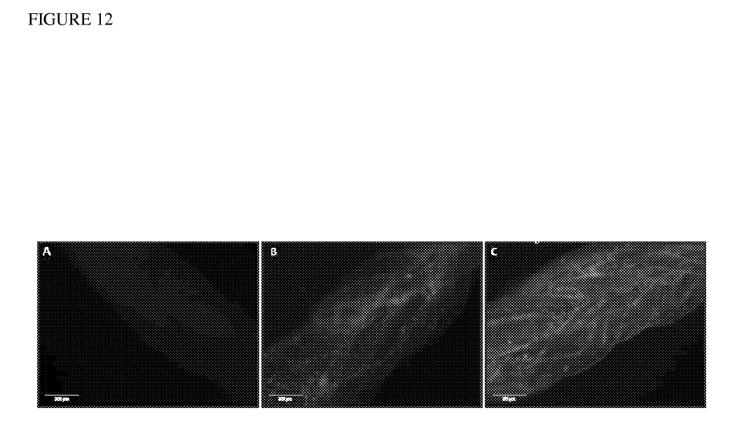

Prior to seeding MDFCs, the cells were preloaded with Mitotracker Green (Invitrogen, Eugene, Oreg.), a mitochondrial dye. The MDFCs were incubated with 500 nM of Mitotracker Green in DMEM for 30 minutes at 37° C. on the day of initial seeding. The cells were washed twice with DPBS and placed back into 37° C. incubation with fresh medium until seeding. To seed MDFCs on braided collagen scaffolds, a PDMS mold was created with an outer circular well with a diameter of 24 mm with two posts in the center creating a channel with a dimension of 2.0 mm by 12 mm (FIG. 11A). This channel was sealed at each end using a thin layer of medical grade silicone adhesive (FIG. 11B). The PDMS mold was sterilized by autoclaving. Next, the silicone adhesive was notched in order to create a wedge to place the braided scaffold through (FIG. 11C). The braided collagen scaffolds were inverted, inserted into the wedge, and sealed into place using sterile vacuum grease (FIG. 11D). The MDFCs in a cell suspension of 200,000 cells in 90 μL of serum free medium (50% DMEM, 50% F-12) were seeded on the scaffolds and incubated for 4 hours at 37° C. (FIG. 11E). Serum free medium was used for the MDFCs attachment to avoid masking the surface biochemical properties of the braided collagen scaffolds. The seeded braided collagen scaffolds were subsequently removed from the PDMS molds, inverted, and placed in a 12-well plate containing culture medium (45% DMEM, 45% F-12, 10% FC III serum) (FIG. 11F). During preliminary studies, uncrosslinked and EDC/NHS crosslinked braided collagen scaffolds were seeded, incubated for 24 hours, and then fixed in 4% paraformaldehyde solution in PBS (USB, Cleveland, Ohio) for 20 minutes at room temperature. The scaffolds were imaged using fluorescence microscopy on an Olympus IX81 motorized inverted microscope coupled to a 12-bit Hamamatzu CCD camera and processed using Slidebook®. The results showed that it was not possible to distinguish or quantify single cells since it is unknown how many mitochondria are inside a MDFC. As such, this labeling method did not allow for quantitative analysis. In addition, further development of the mold needed to be performed to eliminate the detachment of the braided collagen scaffolds from the PDMS ring during insertion into the channel wedge and to optimize the number of cells attaching to the scaffold.

In the first stage of developing a reproducible cell seeding method that allowed for quantitative analysis of MDFCs on braided collagen scaffolds, MDFCs were preloaded with Mitotracker Green and seeded onto scaffolds using a mold with a seeding channel of 2.0 mm×12.0 mm. Following the protocol, it was found that sealing the channel with the silicone adhesive caused a fraction of the braided collagen scaffolds to break from the PDMS ring while trying to insert the braid into the wedge opening. Although the majority of the scaffolds were not damaged when they were inserted into the mold, this iteration of the PDMS cell-seeding mold does not allow for sufficient reproducibility. Compared to the unseeded braided collagen scaffold (FIG. 12A), it appears that using the PDMS mold to seed the braided collagen scaffolds is successful since a fluorescent signal appears on the seeded scaffolds. Fluorescence microscopy images of uncrosslinked (FIG. 12B) and crosslinked (FIG. 12C) seeded braided collagen scaffolds show that seeding with the cell suspension method ensures that the entire braid is exposed to the MDFCs. The results show that MDFCs attached predominately in the grooves of the braids. By visual inspection, it appeared that the uncrosslinked and crosslinked braided collagen scaffolds had similar seeding efficiencies. Unfortunately, fluorescently tagging the mitochondria within the MDFCs only allowed for qualitative analysis because the mitochondria stain did not facilitate discrete cell counting, so cell attachment differences between uncrosslinked and crosslinked scaffolds could not be analyzed. In addition, the large amount of void space in the seeding channel may not facilitate a high seeding efficiency.

### Optimizing the Cell Seeding Method

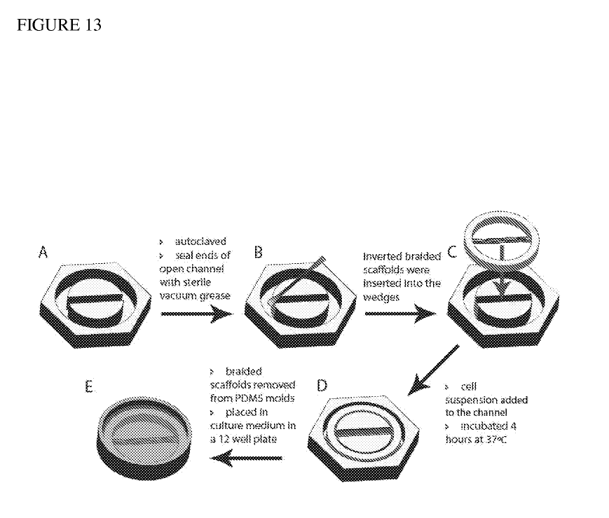

To overcome the limitations associated with using Mitotracker Green prior to seeding on the braided collagen scaffold, MDFCs were incubated with 5 μg/mL Hoechst dye (Invitrogen, Carlsbad, Calif.) in culture medium for 15 minutes at 37° C. on the day of initial seeding. The cells were washed twice with DPBS and placed back into 37° C. incubation with fresh medium until seeding. To determine the optimal environment for uniform and reproducible seeding, the channel within the PDMS mold designed with dimensions of either 2.0 mm by 12 mm or 1.0 mm by 12 mm (FIG. 13A). A smaller channel width was used to eliminate the void space around the scaffold when inserted into the channel. The molds were sterilized by autoclaving, and the channels were sealed at the ends using a thin layer of sterile vacuum grease (FIG. 13B). The sterile braided collagen scaffolds were inverted and inserted into the vacuum grease such that the braid lies on the bottom of the channel (FIG. 13C). The MDFCs were seeded on the scaffolds by adding a cell suspension in serum free medium (50% DMEM, 50% F-12) to the channel containing the braided scaffolds and incubated for 4 hours at 37° C. (FIG. 13D). A cell suspension of 200,000 cells in 90 μL was used for the 2.0 mm by 12 mm channel, and a suspension of 150,000 cells in 30 μL, was used for the 1.0 mm by 12 mm channel (Table 3). Different seeding volumes were used since 90 μL of solution exceeded the volume of the smaller channel. The seeded braided scaffolds were removed from the PDMS molds and placed in a 12-well plate containing culture medium (45% DMEM, 45% F-12, 10% FC III serum) supplemented with 1% pen/strep (vol/vol) (FIG. 13E). The seeded scaffolds were incubated at 37° C. Uncrosslinked and EDC/NHS crosslinked braided collagen scaffolds were seeded, incubated for 24 hours, and then fixed in 4% paraformaldehyde solution in PBS (USB, Cleveland, Ohio) for 20 minutes at room temperature. The scaffolds were imaged using fluorescence microscopy with an Olympus IX81 motorized inverted microscope coupled to a 12-bit Hamamatzu CCD camera and processed using Slidebook®. Due to better seeding uniformity and more reproducible data, the PDMS mold with a channel dimension of 1.0 mm by 12 mm was used in all subsequent experiments.

TABLE 3
Preliminary Cell Seeding Method Development

| Channel Dimension | # Cells Initially Seeded | Seeding Volume |
|---|---|---|
| 2.0 mm × 12 mm | 200,000 | 90 μL |
| 1.0 mm × 12 mm | 150,000 | 30 μL |

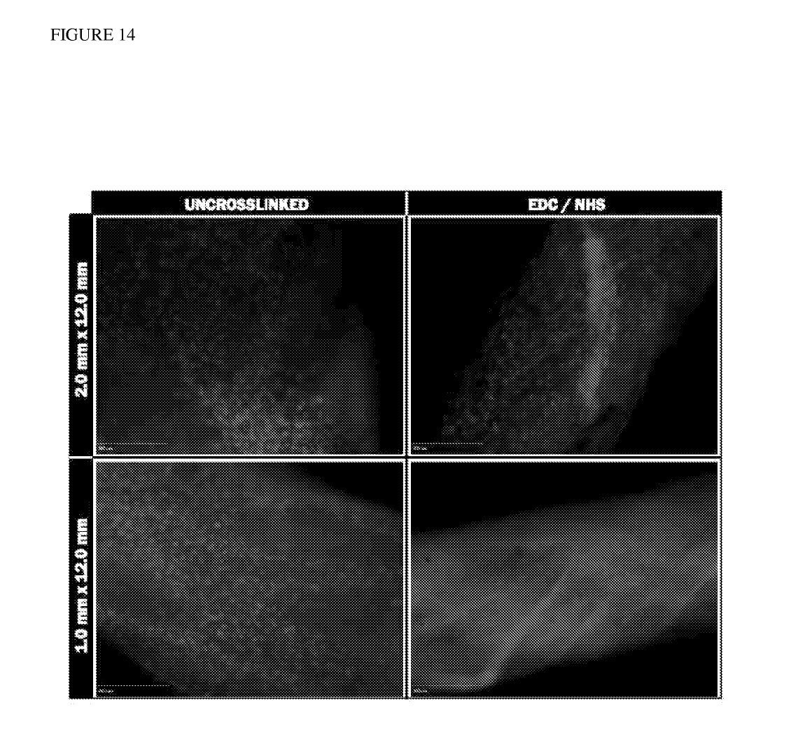

In order to rectify the limitations discovered in the analysis of the preliminary cell seeding results, MDFCs were loaded with Hoechst dye prior to seeding onto braided collagen scaffolds instead of Mitotracker green. MDFCs were seeded onto uncrosslinked and crosslinked braided collagen scaffolds using PDMS molds with channel widths of either 2.0 mm or 1.0 mm, and the results are summarized in Table 4. Using a channel sealed with sterile vacuum grease resulted in elimination of scaffold breakage, but there was still a risk of the cell suspension leaking out of the ends of the channel. Since more than fifty percent of the braided collagen scaffolds seeded in both PDMS mold channel types, this was not considered a significant problem. Hoechst stained images of uncrosslinked and crosslinked braided collagen scaffolds seeding using the two channel widths are shown in FIG. 14. These images do not show large visual difference between the surface treatments due to limitations in imaging a three-dimension scaffold, but in contrast to scaffolds preloaded with Mitotracker green, individual cells can be distinguished from one another enabling quantitative analysis. It is apparent that the MDFCs attached onto the scaffolds seeded in the 1.0 mm wide channel more uniformly with a clear increase in cell number compared to the scaffolds seeded using the wider channel.

TABLE 4
Cell seeding optimization summary table comparing different seeding channel dimensions

| | | Uncrosslinked | EDC/NHS |
|---|---|---|---|
| 2.0 mm × 12.0 mm | Sample Size | 3 | 3 |
| | Total Scaffolds Successfully Seeded | 66.7 | 100 |
| | (%) | | |
| | MDFC Attachment | 23.4 ± 0.70 | 29.9 ± 1.2 |
| | (# of cells/10,000 μm² ± SEM) | | |
| | Total MDFC attachment | 23,501 ± 704.6 | 30,087 ± 1,230 |
| | (# cells ± SEM) | | |
| | Total MDFCs Successfully Seeded | 11.8 ± 0.4* | 15.0 ± 0.6* |
| | (% ± SEM) | | |
| 1.0 mm × 12.0 mm | Sample Size | 4 | 4 |
| | Total Scaffolds Successfully Seeded | 100 | 75 |
| | (%) | | |
| | MDFC Attachment | 31.1 ± 1.12 | 36.1 ± 0.91 |
| | (# of cells/10,000 μm² ± SEM) | | |
| | Total MDFC attachment | 31.277 ± 1,038 | 36.356 ± 914.8 |
| | (# cells ± SEM) | | |
| | Total MDFCs Successfully Seeded | 20.9 ± 0.7 | 24.2 ± 0.6 |
| | (% ± SEM) | | |

*Indicates statistically significant differences between 2.0 mm wide channels and 1.0 mm wide channels with p < 0.05 using Kruskal-Wallis One Way ANOVA on Ranks with Dunn's Method.

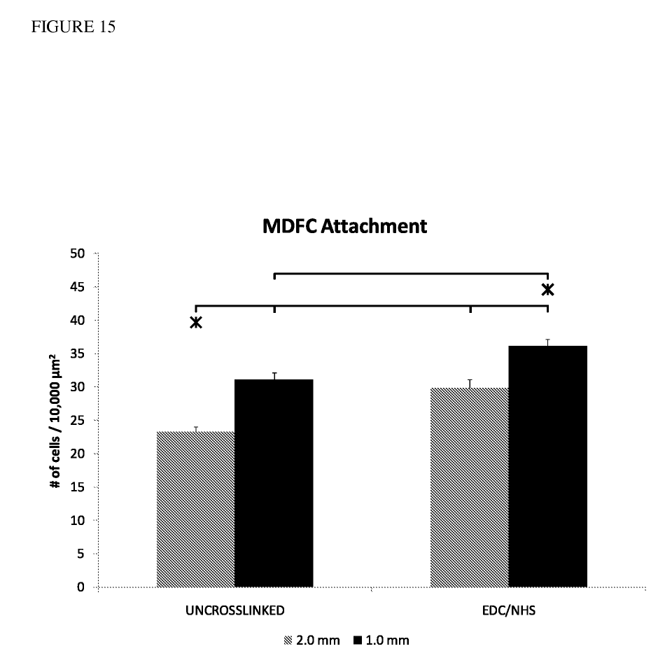

The density of cells that attached to the braided collagen scaffolds in an area of 10,000 μm² were counted visually to compare the two different seeding methods. Seeding with either the 2.0 mm wide channel or the 1.0 mm wide channel showed a significant increase in cell density between uncrosslinked and crosslinked scaffolds (FIG. 15). The results show seeding using the narrower channel significantly increased the seeding density on both uncrosslinked and crosslinked scaffolds compared to the wider channel.

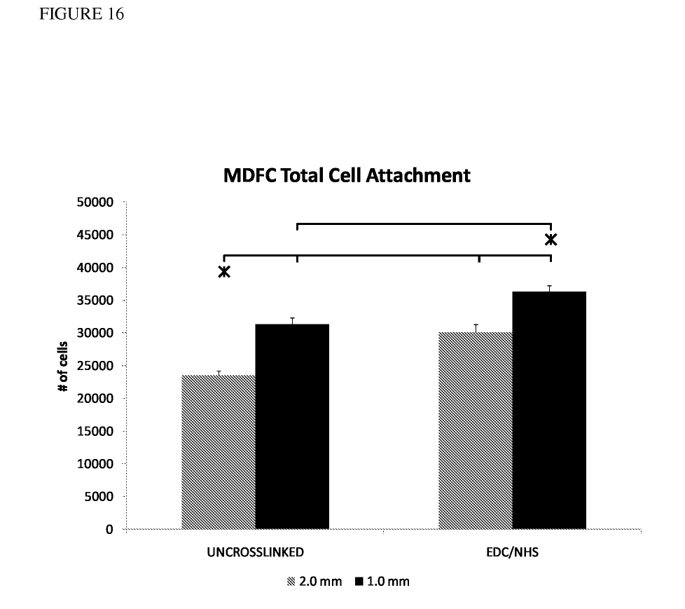

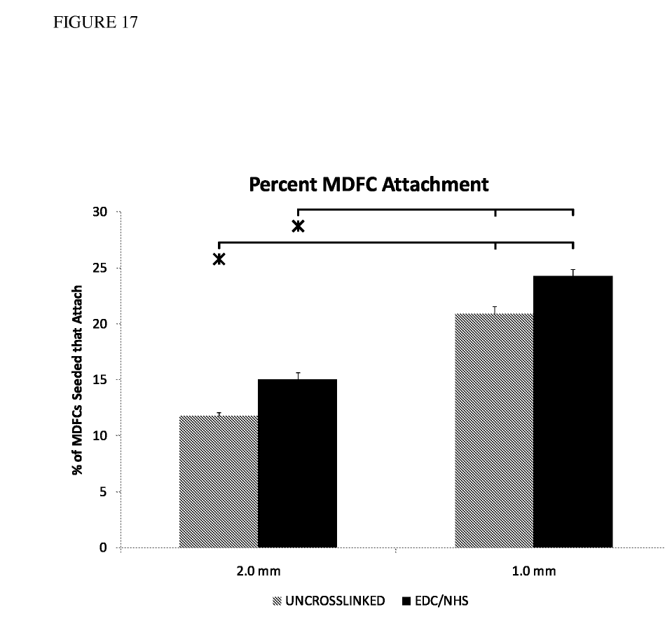

The total number of MDFCs that attached to the braided collagen scaffold was approximated in order to determine which channel width resulted in the better seeding efficiency. As expected from the regional cell attachment counts, seeding with the narrower channel caused significantly more cells to attach to the surface compared to the wider channel with at least 6,000 more cells attached in both uncrosslinked and crosslinked scaffolds (FIG. 16). Since the channels were seeded with different cell suspension concentrations to adjust for the different channel volumes, the total cell attachment was normalized by calculating the percentage of MDFCs used in the cell suspension that actually seeded (FIG. 17). Using the narrower channel to seed MDFCs resulted in 20-25% of the cells in the suspension attaching to the braided collagen scaffold, which was significantly higher than the wider channel, which resulted in less than 15% attachment on each surface modification.

Two channel widths, 1.0 mm and 2.0 mm, were examined to determine which resulted in the highest seeding efficiency. It was found that using a channel width of 1.0 mm resulted in 21% and 24% of the MDFCs attaching to the uncrosslinked and EDC/NHS crosslinked braids, respectively since it had less void space around the braid in the channel. A limitation of this novel MDFC seeding method is the braids are not exposed consistently to cells throughout the entire surface area. When seeding within the channel, it is not easy to control how the braids lies on the bottom of the PDMS mold, so if the braid is touching the bottom, the cells in the suspension are unable to flow under the braid to attach. This resulted in an influx of cells on either side of the braided threads in the channel. If the braid is lifted slightly from the bottom of the PDMS mold, then cells are able to flow under the scaffold, exposing the underside of the braid to cells. There was no difference in seeding uniformity and concentration between scaffolds that seeded on the whole surface area and ones that only seeded on half.

There are several ways to overcome this limitation during the cell seeding process. Cornwell et al. seeded bundles of fibrin threads by exposing a cell suspension to the threads on a Thermanox® square, but since braided collagen threads are much larger and more structurally dense, using this method resulted in the braid drying out after 30 minutes. Altman et al. used a Teflon seeding chamber similar to the PDMS mold used in this study to seed their silk fiber cords. Instead of exposing one area of the cords to the cell suspension, the cords were rotated 90 degrees while adding additional cells to the chamber until the entire cord was exposed to cells. Using the Teflon seeding chamber, the seeding efficiency was approximately 10%, but since cells have a greater affinity for collagen and using longer cell suspension exposure times, the efficiency can be increased during the seeding period. To create the effect of physically rotating the scaffold in the chamber, a bioreactor can be utilized that will rotate the chamber around the scaffold at a controlled rate.

A second limitation to overcome was how to visually characterize the MDFCs on the threads. It was observed that using a Mitrotracker dye was sufficient to visualize cells attached to the braids, but it was not possible to quantify discrete cells since it is difficult to correlate mitochondria with cell numbers. Fluorescently tagging MDFCs with Hoechst dye prior to seeding onto braided collagen scaffolds allowed for quantification of MDFCs and was used for the remainder of the experiments in this study. Limitations of using Hoechst dye are that time lapse experiments using the same scaffold is not possible since exposing the Hoechst loaded cells to ultraviolet light activates the dye, which can cause mutations in the DNA, and autofluorescence of collagen between 320 nm and 461 nm can make seeing the cells difficult. Another limitation of using Hoechst dye is that it is unknown if cells expel the dye over a period of time, which could create inconsistencies in cell counts between experiments. It is envisioned that one way to overcome these limitations on the braids would be to express green fluorescent protein (GFP) in the MDFCs because in this excitation range, the collagen is less autofluorescent. In addition, using GFP will allow cells to be visualized on braids for time-lapse experiments.

Quantifying Cell Attachment and Growth on Collagen Braided Scaffolds with Different Surface Modifications

FGF-2 modified surfaces promote MDFC attachment and growth on braided collagen scaffolds. Braided collagen scaffolds that were uncrosslinked, EDC/NHS crosslinked, EDC/NHS crosslinked with heparin, and EDC/NHS crosslinked with heparin with 5 ng/mL, 10 ng/mL, or 50 ng/mL FGF-2 were seeded with MDFCs that were preloaded with Hoechst dye. After culture for 1, 5, or 7 days, the scaffolds were removed and analyzed both quantitatively and qualitatively. Using image J software, the number of Hoechst stained nuclei per 10,000 μm² was counted to determine cell density and cell distribution. In addition, scaffolds were stained with phalloidin, a fluorescent stain that binds to the f-actin filaments, to characterize the cellular alignment of MDFCs on braided collagen scaffolds.

### Quantification of Cell Number on Braided Collagen Threads

### Cell Attachment

To determine the effects of different surface modifications on attachment of MDFCs to braided collagen scaffolds, MDFCs were seeded as described above onto uncrosslinked scaffolds and scaffolds treated with EDC/NHS, EDC/NHS with heparin, or EDC/NHS with heparin coated with FGF-2 at concentrations of either 5 ng/mL, 10 ng/mL, or 50 ng/mL. Unseeded braided collagen scaffolds were used as controls. Seeded braided collagen scaffolds were cultured at 37° C. for 24 hours before fixing in 4% paraformaldehyde solution in PBS (USB, Cleveland, Ohio) for 20 minutes at room temperature. Scaffolds were washed twice for 5 minutes in PBS, and stored in PBS at 4° C. until imaging. In order to image the braided scaffolds, they were removed from the PDMS ring and placed on a glass slide covered with enough PBS to maintain hydration throughout the imaging process. For each condition, 8 to 14 scaffolds were imaged from 4 separate experiments by fluorescence microscopy on an Olympus IX81 motorized inverted microscope coupled to a 12-bit Hamamatzu CCD camera and processed using Slidebook®. Imaging locations were chosen for cell quantification at 10× magnification in nonoverlapping focal regions across the entire length of the scaffold by placing it on the slide parallel to the x-axis. This resulted in 5 to 18 images per scaffold depending on the number of focal regions in the z-direction.

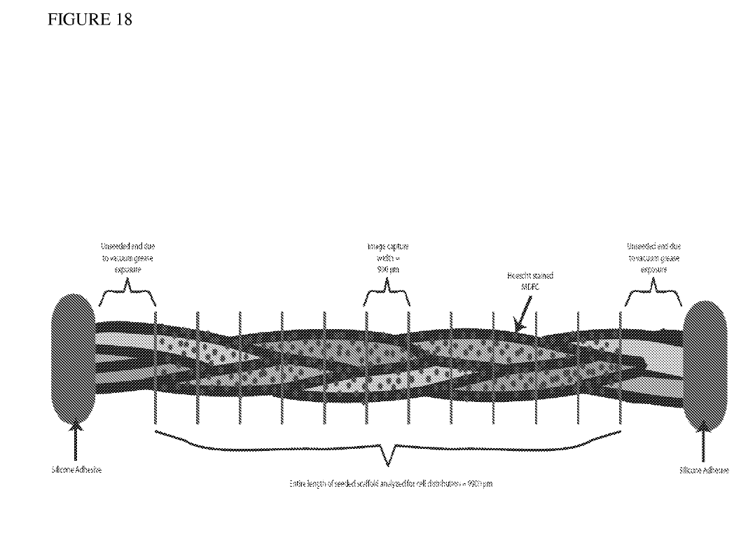

The images were analyzed using Image J software with the grid and cell counter plug-in for cell attachment and cell distribution across the length of the scaffold. A grid was placed on each image with an area of 10,000 μm² (1.55 pixels/μm) between each grid line. Using the cell counter plug-in, raw data was collected from each image as the average number of Hoechst dye stained nuclei counted in four separate regions. Not all of the cells are in the focal plane of the each image because of the limited focal depth when imaging three-dimensional scaffolds. As such, the data was normalized by reporting it as the number of cells within an area of 10,000 μm². To determine whether there was an equal cell distribution across length of the braided collagen scaffold, cells were counted using the procedure described above for images taken every 900 μm along the length of the scaffold (FIG. 18). Although the majority of the scaffolds were seeded over the entire surface area, for the purposes of this cell attachment assay and cell distribution, only one side of the braided scaffold was analyzed. There was no difference in seeding throughout the surface area of the scaffold.

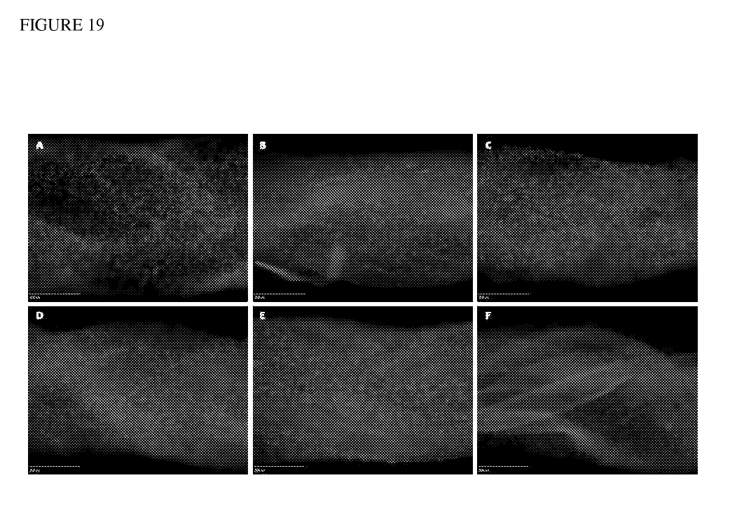

In order to determine the effects of surface modifications on MDFC attachment, braided collagen scaffolds with different surface modifications were seeded with MDFCs and incubated for 24 hours. The results of the cell attachment assay are summarized in Table 5. Fluorescence images of the Hoechst dye stained braided collagen scaffolds are shown in FIG. 19. The MDFCs seeded uniformly spread over the entire surface of the braided collagen scaffold. The images show a clear increase in cell attachment from the uncrosslinked scaffold surface (FIG. 19A) to the crosslinked and FGF-2 bound scaffold surfaces. The EDC/NHS HEP (FIG. 19C) braided collagen scaffold appears to have a higher density of cells compared to the EDC/NHS (FIG. 19B), 5 ng/mL FGF-2 (FIG. 19D), 10 ng/mL FGF-2 (FIG. 19E), and 50 ng/mL FGF-2 (FIG. 19F) braided collagen scaffolds.

TABLE 5
Cell Attachment summary table comparing different surface modifications

| | | MDFC Attachment |
|---|---|---|
| | Sample Size | (# of cells/10,000 μm² ± SEM) |
| UNCROSSLINKED | 13 | 34.9 ± 0.69* |
| EDC/NHS | 13 | 41.6 ± 0.76 |
| EDC/NHS HEP | 11 | 46.5 ± 0.99† |
| 5 ng/mL FGF2 | 12 | 40.3 ± 0.73 |
| 10 ng/mL FGF2 | 8 | 39.2 ± 0.70 |
| 50 ng/mL FGF2 | 8 | 40.7 ± 0.99 |

*Indicates statistically significant differences between uncrosslinked and all other conditions with p < 0.05 using Kruskal-Wallis One Way ANOVA on Ranks with Dunn's Method.
†Indicates statistically significant differences between uncrosslinked and all other conditions with p < 0.05 using Kruskal-Wallis One Way ANOVA on Ranks with Dunn's Method.

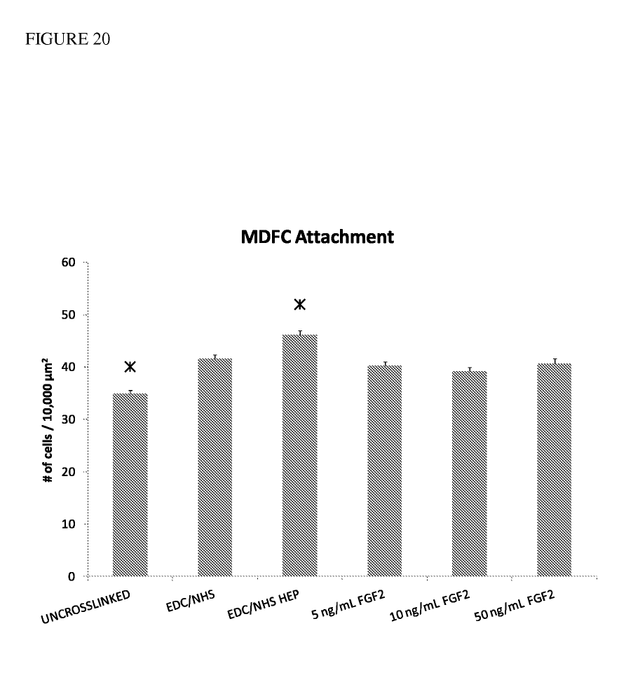

The density of cells that attached to the braided collagen scaffolds in an area of 10,000 μm² were counted visually to compare how surface modifications affected cell attachment (FIG. 20). There was a significant increase in cell attachment from uncrosslinked braided collagen scaffolds to braids with surface modifications. Braided collagen scaffolds that were EDC/NHS crosslinked with heparin promoted a significantly higher cell attachment to its surface than all other scaffold surfaces. Increasing the amount of FGF-2 bound to the surface does not significantly affect cell attachment. There was not a significant difference in cell attachment between EDC/NHS crosslinked scaffolds and all scaffolds with FGF-2 bound to the surface.

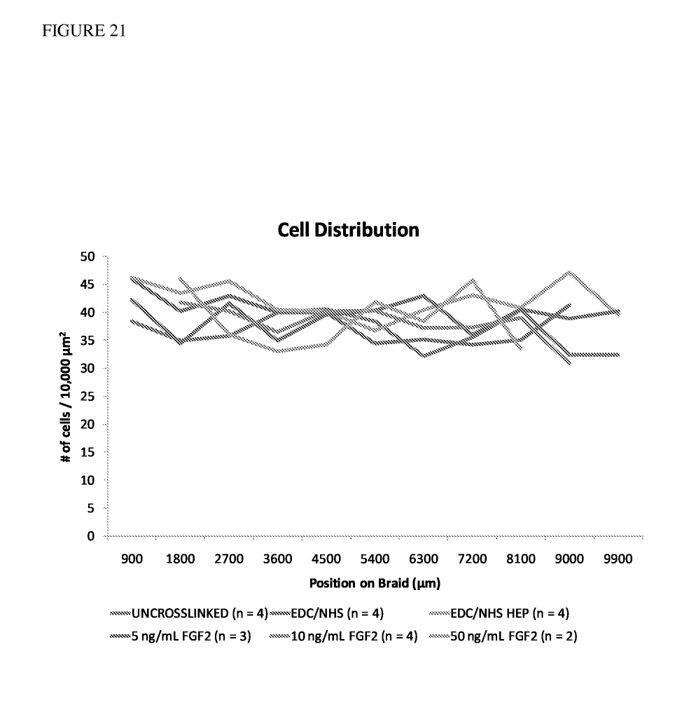

To determine if seeding using the PDMS mold distributes the MDFCs uniformly across the entire length of the scaffold, images were taken in adjacent regions across the entire length of the braids and cells were counted. The results are reported as the average number of cells per 10,000 μm² for every 900 μm across the scaffolds on the x-axis (FIG. 21). These results show that using the PDMS mold to seed the braided collagen scaffolds resulted in uniform distribution across the length of the braid, with approximately 7,392±1,669 μm of the scaffold being exposed to cells on average. Although only one side of the scaffold was analyzed for cell distribution, it was apparent from visual analysis that the majority of the scaffolds seeded uniformly across the entire surface area.

### Cell Growth

To determine the effects of different surface biochemistries on the growth of MDFCs on braided collagen scaffolds, MDFCs preloaded with Hoechst dye were seeded as described above onto uncrosslinked scaffolds and scaffolds treated with EDC/NHS, EDC/NHS with heparin, and EDC/NHS with heparin coated with FGF-2 at concentrations of either 5 ng/mL, 10 ng/mL, or 50 ng/mL. Unseeded scaffolds were used as controls. Seeded braided collagen scaffolds were cultured at 37° C. moving the scaffolds to a new sterile 12 well plate with fresh medium every other day to prevent contamination during extended culture periods. Scaffolds were cultured for 5 days and 7 days before fixing in 4% paraformaldehyde solution in PBS for 20 minutes at room temperature. Scaffolds were washed twice for 5 minutes in PBS, and stored in PBS at 4° C. until imaging. Seeded scaffolds cultured for 5 and 7 days were analyzed for cell growth in the same manner as described previously for cell attachment and cell distribution.

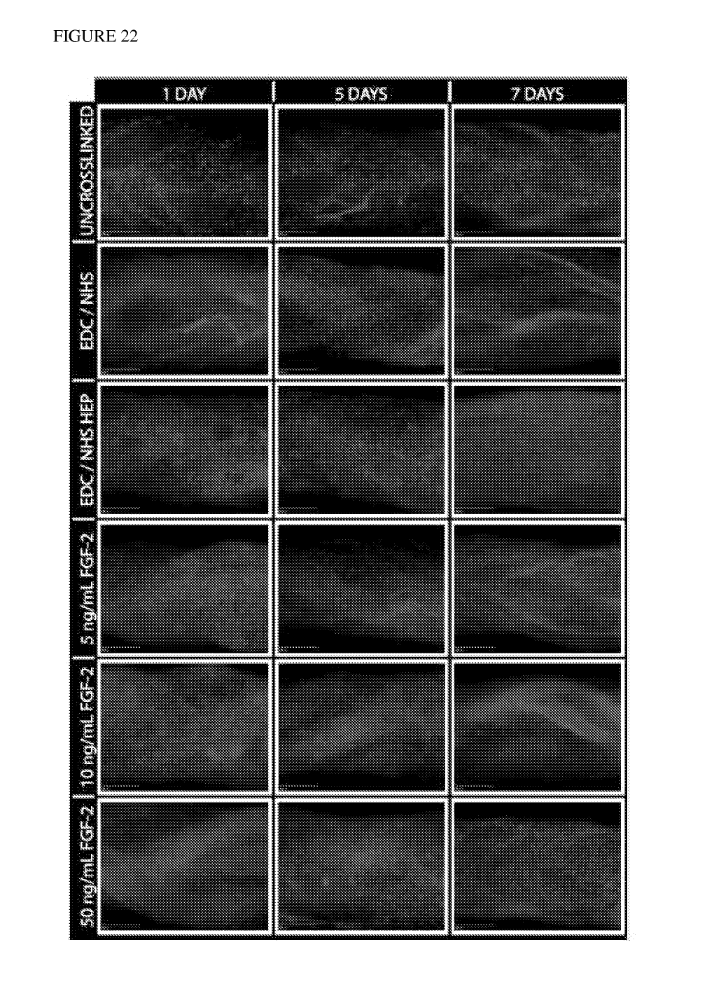

The effect of binding FGF-2 on MDFC growth and proliferation was determined by seeding MDFCs and incubating them on the braided collagen scaffolds for 1 day, 5 days, or 7 days. The results of the cell growth assay are summarized in Table 6. Fluorescence images of Hoechst dye stained braided collagen scaffolds are shown in FIG. 22. During incubation, MDFCs seeded uniformly showing a minor increase in cell concentration in the grooves of the braid topography, and by the seventh day, cells have completely spread out to cover the surface of the braid. All braided scaffolds showed an increase in cell density from 1 day to 7 days showing most of the growth happening between 5 and 7 days. The greatest overall cellular growth appears to occur within the scaffolds with FGF-2 bound to the surface.

TABLE 6
Cell growth summary table comparing different surface modifications

| | 1 DAY | | 5 DAYS | | 7 DAYS | |
|---|---|---|---|---|---|---|
| | Sample Size | $\frac{\# of cells}{10 , 000 {µm}^{2}} \pm SEM$ | Sample Size | $\frac{\# of cells}{10 , 000 {µm}^{2}} \pm SEM$ | Sample Size | $\frac{\# of cells}{10 , 000 {µm}^{2}} \pm SEM$ |
| UNCROSSLINKED | 13 | 34.9 ± 0.69 | 10 | 37.0 ± 0.91 | 10 | 47.7 ± 0.89† |
| EDC/NHS | 13 | 41.6 ± 0.76 | 10 | 44.1 ± 0.79† | 9 | 50.8 ± 1.07† |
| EDC/NHS HEP | 11 | 46.5 ± 0.99 | 9 | 46.2 ± 0.75 | 9 | 53.3 ± 1.26* |
| 5 ng/mL FGF-2 | 12 | 40.3 ± 0.73 | 9 | 47.1 ± 0.77* | 9 | 55.6 ± 1.18* |
| 10 ng/mL FGF-2 | 8 | 39.2 ± 0.70 | 10 | 42.5 ± 0.71* | 10 | 63.5 ± 1.36†† |
| 50 ng/mL FGF-2 | 8 | 40.7 ± 0.99 | 8 | 44.8 ± 0.91 | 9 | 73.2 ± 1.63†† |

*Indicates statistically significant differences between the growth of that surface modification at that day and the growth at all previous days with p < 0.05 using Kruskal-Wallis One Way ANOVA on Ranks with Dunn's Method.
†Indicates statistically significant differences between the growth of that surface modification at that day and the growth at all previous days with p < 0.05 using One Way ANOVA with Holm-Sidak method.
††Indicates statistically significant differences between 10 ng/mL FGF-2 and 50 ng/mL FGF-2 and all other modifications at 7 days as well as statistically significant differences between the cell growth of these modifications at 7 days and the growth at 1 day and 5 days with p < 0.05 using Kruskal-Wallis One Way ANOVA on Ranks with Dunn's Method.

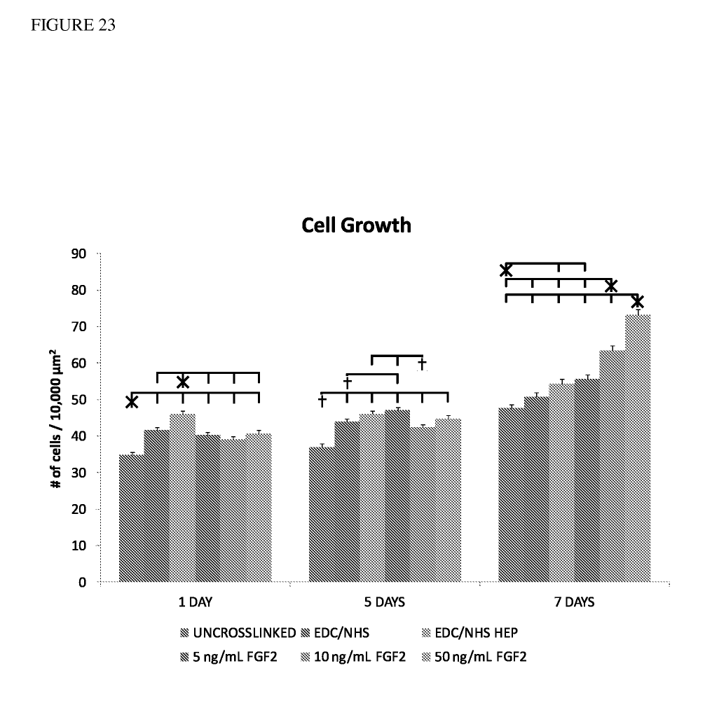

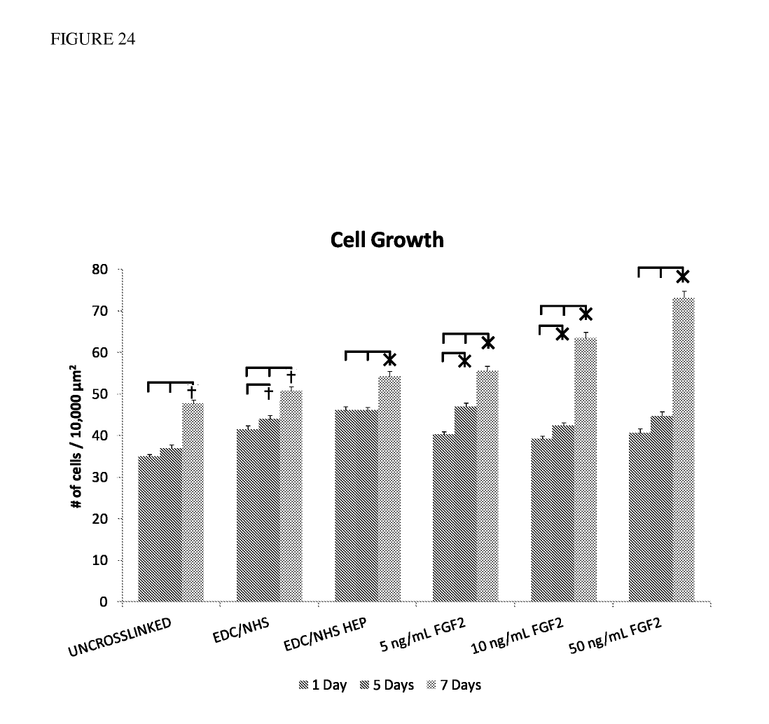

The density of cells that attached to the braided collagen scaffolds in an area of 10,000 μm² were counted visually to compare how surface modifications affected cell growth after 7 days in culture (FIG. 23). The concentration of MDFCs on the surface of the braids increased on both control and modified braids between day 1 and day 7. After 5 days in culture, the number of cells on each of the braid types did not increase significantly except for cells attached to braids modified with EDC/NHS crosslinking and 5 ng/mL and 10 ng/mL of FGF2. Uncrosslinked scaffolds had significantly fewer cells on the surface than all other scaffold types, and scaffolds modified with 5 ng/mL FGF-2 had a significantly higher cell densities than all other braids except types modified with EDC/NHS and heparin and 50 ng/mL FGF-2. By day 7, all braided collagen scaffolds showed a significant increase in cell concentration compared to day 1. In addition, between day 5 and day 7, scaffolds modified with different concentrations of FGF-2 showed a significant increase in cell growth compared to the controls with increasing levels of FGF-2 (FIG. 24).

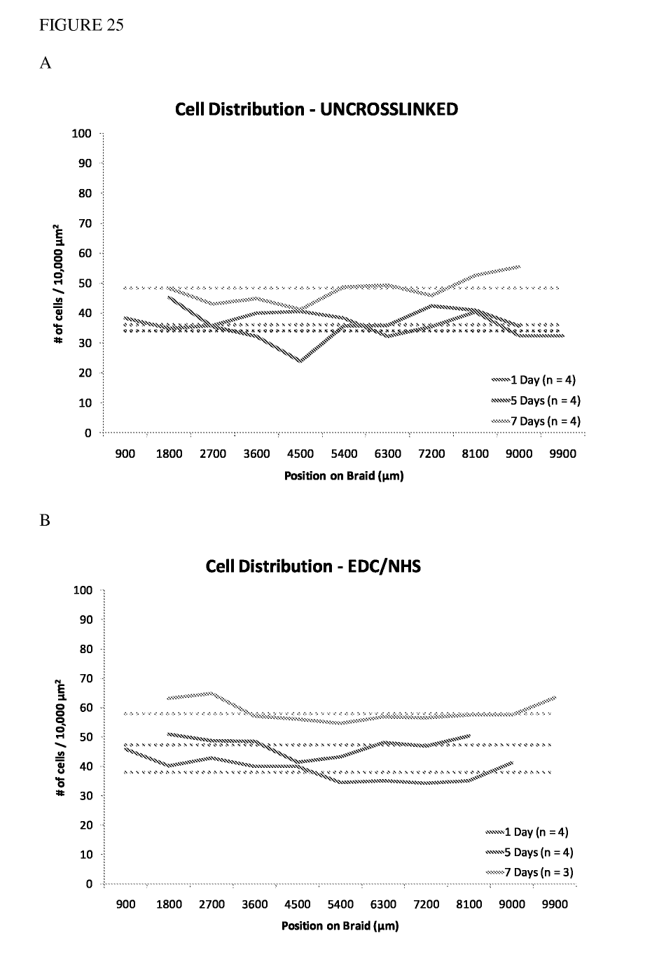

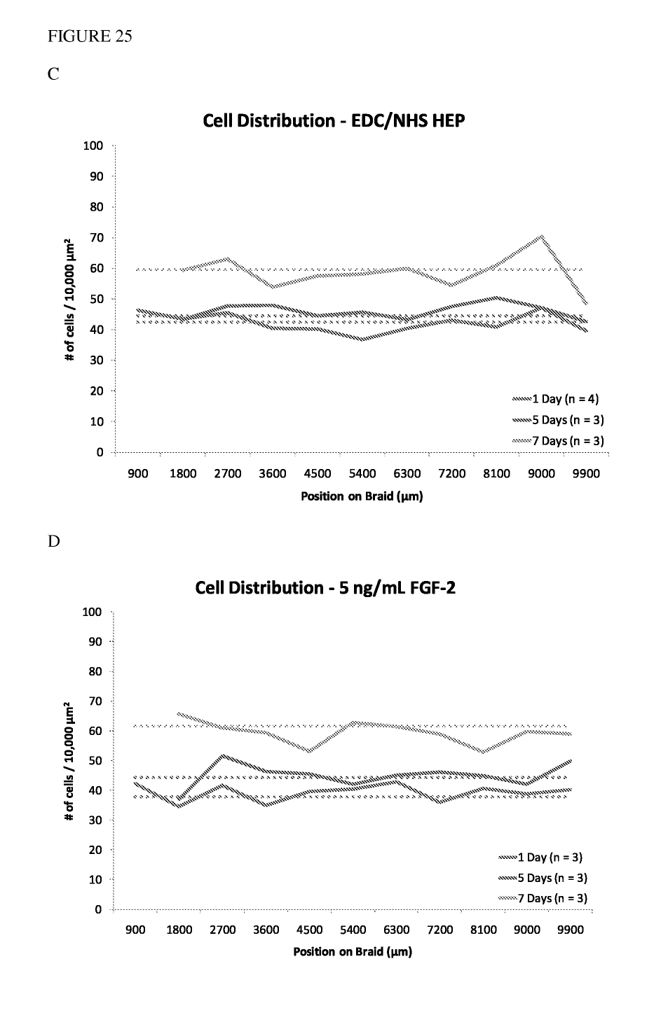

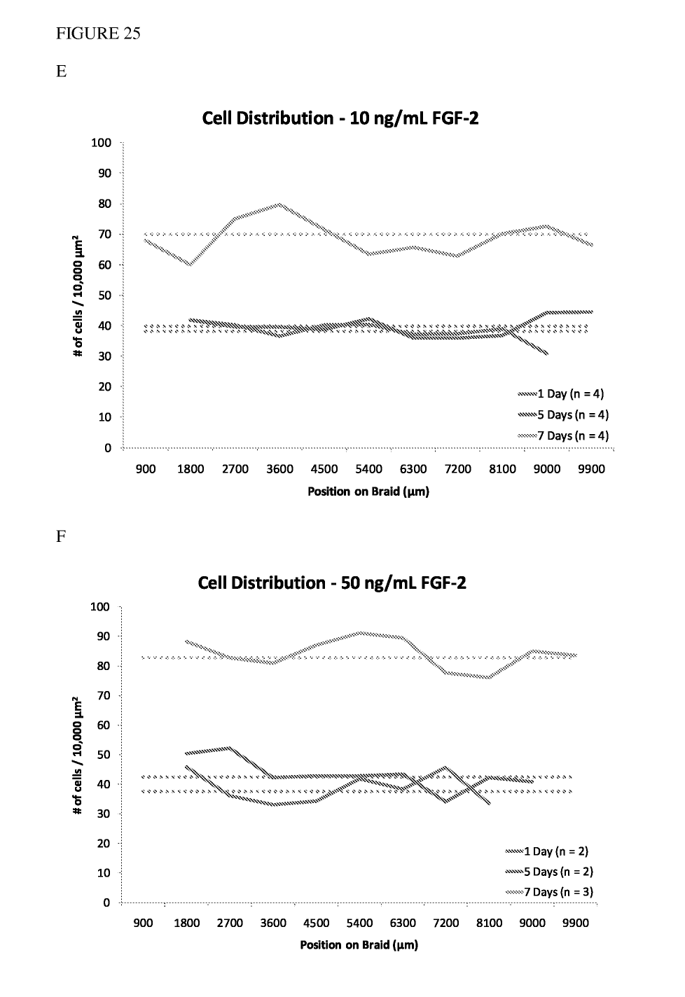

The cell distribution data for the cell growth over 7 days on the different surface modifications shows the cells grow evenly along the length of the scaffold (FIG. 25). The trend of the distribution lines (solid) fluctuate around the average cell growth (dashed) for each braided scaffold with minimal changes between 1 and 5 days. Interestingly, scaffolds loaded with 10 ng/mL and 50 ng/mL FGF-2 shows a statistically significant difference in growth from 5 to 7 days.

### Estimation of Total Cell Attachment and Growth

The cross sectional perimeter was established using the histological sections of three hematoxylin and eosin stained unseeded braided collagen threads. The outer edge of the scaffolds was traced using Image J software in order to obtain an approximate surface perimeter. To account for the differences in surface topography on the scaffold, sections were measured at four different locations along the length of the scaffold and averaged together. When the braided collagen scaffold is placed inside the PDMS mold, the ends of the scaffold are exposed to sterile vacuum grease, which prevents MDFCs from attaching beyond this boundary. Using the cell distribution data, the length of the seeded area of the braided collagen scaffold can be determined. Using the assumption that all sides have been seeded with MDFCs, the total surface area of the braided collagen scaffold can be determined by multiplying the cross sectional perimeter by the length of the seeded area of the braid. Using this information, the total number of MDFCs attached to the surface at each time point can be extrapolated by multiplying the number of cells counted per 10,000 μm² region by the total seeded surface area. In addition to total cell attachment and growth calculations, the percentage of the cells seeded that attached to the surface and the fold increases of the cells over time was calculated. The increase in cell number over the number of cells that attached, T_{d}, was calculated using the following equation, where q₁ is the average number of cells attached for each surface modification and q₂ is the number of cells at counted at 5 and 7 days.

$${T}_{d} = \frac{{q}_{2}}{{q}_{1}}$$

The total number of cells that attach to the braided collagen scaffolds was determined by multiplying the results in Table 5 by the surface area of an unseeded braided collagen scaffold calculated from histological cross-sections. The cross-sectional perimeter of a braid containing 18 collagen microthreads, which was not significantly different between each surface modification, was found to be 1,361±278 μm. The cross-sectional perimeter was then multiplied by the length of the seeded portion of the braid, which was determined using the cell distribution data, to get a total surface area of 10,059,532±2,058,025 μm². In order to calculate the total attachment, the results in Table 5 were multiplied by 1,006±205.8, which is the surface area divided by 10,000 μm². The results of the total cell attachment are summarized in Table 7.

TABLE 7
Total cell attachment summary table on different surface modifications

| | Total MDFC | Percentage of Attached Cells |
|---|---|---|
| | Attachment | (% of 150,000 cells |
| | (# of cells ± SEM) | seeded ± SEM) |
| UNCROSSLINKED | 35,139 ± 693* | 23.4 ± 0.5* |
| EDC/NHS | 41,893 ± 765 | 27.9 ± 0.5 |
| EDC/NHS HEP | 46,386 ± 913† | 30.9 ± 0.6† |
| 5 ng/mL FGF2 | 40,511 ± 736 | 27.0 ± 0.5 |
| 10 ng/mL FGF2 | 39,414 ± 700 | 26.3 ± 0.5 |
| 50 ng/mL FGF2 | 40,912 ± 1,000 | 27.3 ± 0.7 |

*Indicates statistically significant differences between uncrosslinked and all other conditions with p < 0.05 using Kruskal-Wallis One Way ANOVA on Ranks with Dunn's Method.
†Indicates statistically significant differences between uncrosslinked and all other conditions with p < 0.05 using Kruskal-Wallis One Way ANOVA on Ranks with Dunn's Method.

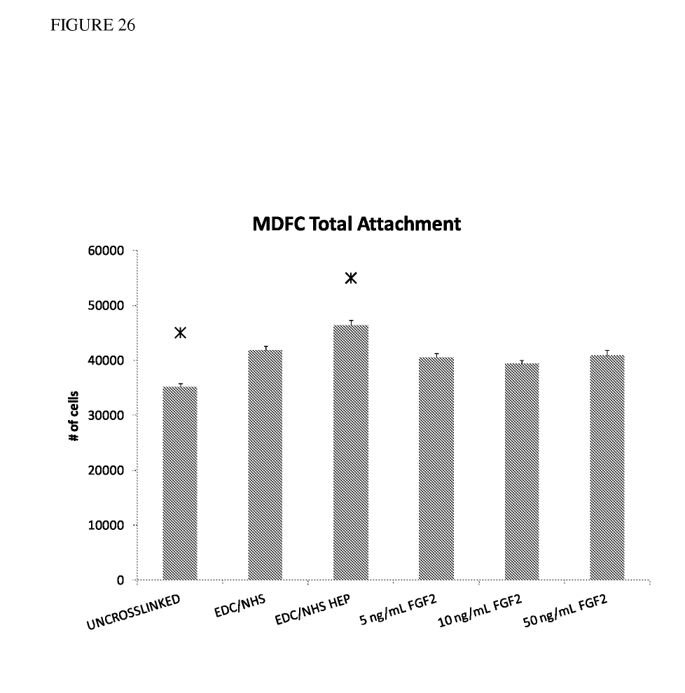

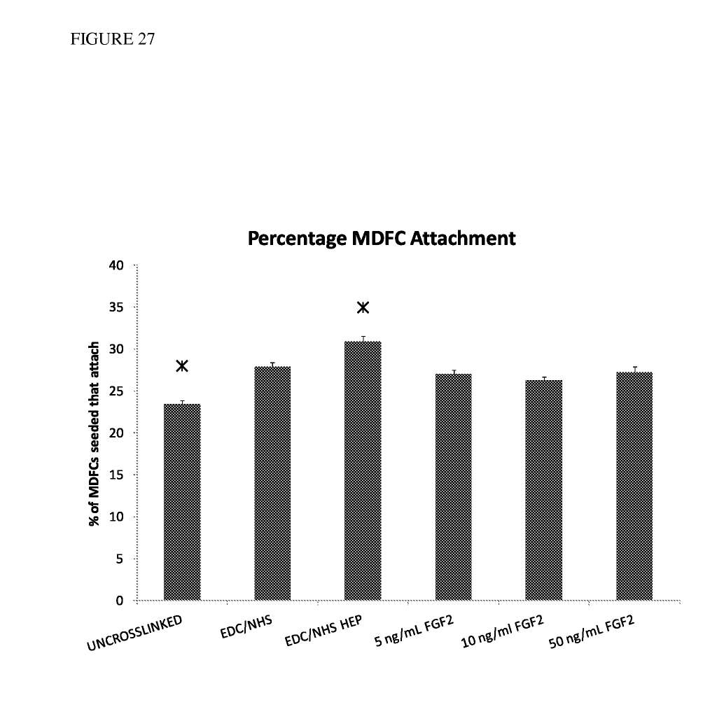

The total number of MDFCs that attached to the braided collagen scaffolds was approximated to determine which surface treatment promoted more cellular attachment. As expected from the regional cell attachment counts, uncrosslinked braided collagen scaffolds promoted significantly less cell attachment than the other braided collagen scaffolds while EDC/NHS with heparin scaffolds promoted significantly more cells to attach (FIG. 26). In order to determine which scaffold had the best seeding efficiency, the percentage of the total amount of cell in the suspension that actually attached to the scaffolds was calculated (FIG. 27). Uncrosslinked braided collagen scaffolds resulted in a significantly lower seeding percentage compared to the other surface modifications with approximately 23% attachment. EDC/NHS with heparin scaffolds resulted in a significantly higher seeding percentage with approximately 31% attachment.

The effect the surface modifications have on the rate of MDFCs growth was determined by extrapolating the doubling time from the total MDFC growth after 5 and 7 days. The results of the total cell growth are summarized in Table 8.

TABLE 8
Total cell growth summary table on different surface modifications

| | 5 DAYS | | 7 DAYS | |
|---|---|---|---|---|
| | Total MDFC Growth | Fold Increase | Total MDFC Growth | Fold Increase |
| | (# of cells ± SEM) | (increase ± SEM) | (# of cells ± SEM) | (increase ± SEM) |
| UNCROSSLINKED | 37,205 ± 918 | 1.06 ± 0.026 | 47,978 ± 893 | 1.37 ± 0.025 |
| EDC/NHS | 44,337 ± 794 | 1.06 ± 0.019 | 51,123 ± 1,080 | 1.22 ± 0.026 |
| EDC/NHS HEP | 46,670 ± 753 | 1.00 ± 0.017 | 54,670 ± 1,265 | 1.18 ± 0.027 |
| 5 ng/mL FGF-2 | 47,399 ± 770 | 1.17 ± 0.019 | 55,977 ± 1,187 | 1.38 ± 0.029 |
| 10 ng/mL FGF-2 | 42,742 ± 712 | 1.08 ± 0.018 | 63,907 ± 1,371* | 1.62 ± 0.035* |
| 50 ng/mL FGF-2 | 45,094 ± 916 | 1.10 ± 0.022 | 73,610 ± 1,639* | 1.80 ± 0.040* |

*Indicates statistically significant differences between the growth/increase over attachment of MDFCs at 7 days for 10 ng/mL and 50 ng/mL FGF-2 and all other surface modifications with p < 0.05 using Kruskal-Wallis One Way ANOVA on Ranks with Dunn's Method.

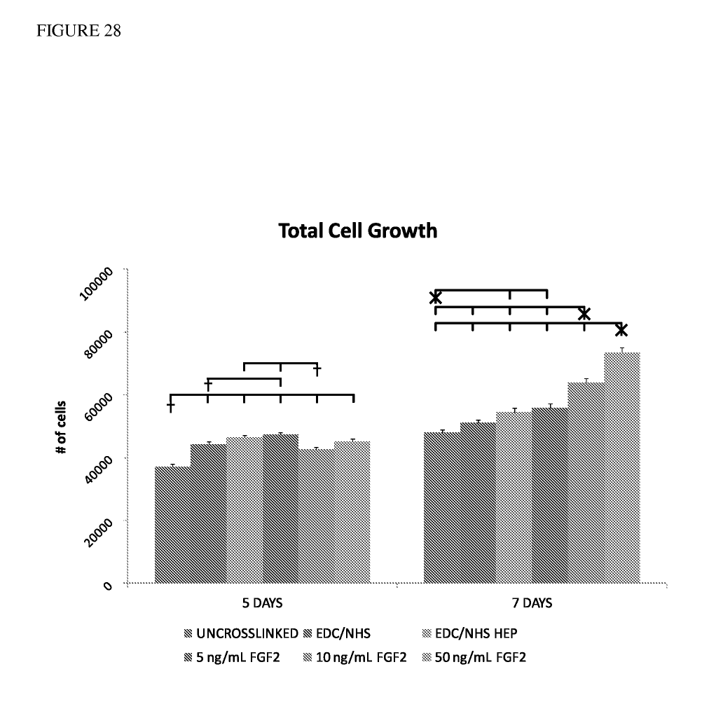

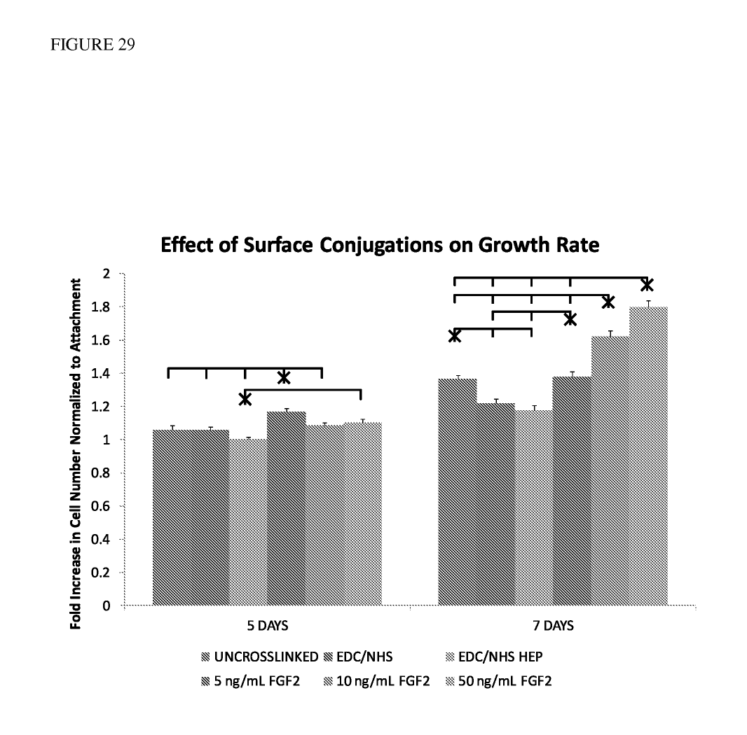

The total number of MDFCs present on the braided collagen scaffold after 5 and 7 days in culture, was approximated to determine which surface modification promoted the highest growth rate. As expected from the regional cell growth counts, after 5 days in culture, none of the modified braid types promoted significant cell growth relative to the day 1 attachment data expect braids with 5 ng/mL FGF-2 bound to the surface. However, there was significant growth after 7 days, with surfaces modified with 50 ng/mL FGF-2 having approximately 74,000 cells present, which was significantly higher than all other conditions (FIG. 28). To determine which scaffold promoted the highest growth rate, the fold increase in cell number normalized to the average cell attachment for each condition was calculated (FIG. 29). After 5 days, all control surface modifications as well as scaffolds modified with 10 ng/mL FGF-2 had significantly less growth than surfaces modified with 5 ng/mL FGF-2. After 7 days, the growth rate on EDC/NHS crosslinked scaffolds and EDC/NHS crosslinked with heparin scaffolds was significantly less than all other conditions, and scaffolds modified with 10 and 50 ng/mL FGF-2 had significantly higher growth rates than all other scaffold types.

Studies show that FGF-2, which is located in the basal lamina surrounding myofibers, is upregulated during skeletal muscle regeneration and is involved in the proliferation and fusion of developing myofibers. Due to the angiogenic properties of the protein, FGF-2 also plays a role in revascularizing the defect during the inflammatory and degradation stages of muscle wound healing. During in vivo studies, the injection of FGF-2 into injured muscle expedited the wound healing process, decreasing the formation of scar tissue and increasing function and movement of the muscle. The teachings of this disclosure show the effects of FGF-2 and heparin on MDFCs attachment, which was analyzed by seeding MDFCs to braided collagen scaffolds with different surface modifications and observing the cellular attachment after 24 hours in culture. The results show a significant decrease in cellular affinity for uncrosslinked braided collagen scaffolds, and a significant increase in cellular affinity for EDC/NHS crosslinked with heparin braided collagen scaffolds. The difference in cell attachment between each of the FGF-2 bound scaffolds and the EDC/NHS crosslinked scaffolds were not significantly different.

In vitro studies comparing uncrosslinked controls with scaffolds with surface modifications, specifically heparin, show no difference in attachment on collagen scaffolds, which is inconsistent with our findings that heparin promotes higher MDFC attachment compared to all other surface treatments. In the teachings of this disclosure, heparin promoted significantly higher attachment of MDFCs than all other braided collagen scaffold surfaces. One variation that could account for this difference is that braided collagen scaffolds were prepared for cell attachment without exposure to serum, which affects the interaction between the cell signals and the modifications on the surface. In addition, the cell seeding protocol was conducted without the addition of serum to the culture medium, in order to ensure the most accurate reflection on cell affinity for the different surface modifications. Another difference that could cause the inconsistencies between the previous observations and the current studies would be the seeding method. Wissink et al. loaded collagen sponges with different surface modifications by adding the cell suspension directly onto the surface, which eliminates attachment differences between the surface modifications. Another study using fibrin threads exposed the bundles to fibroblast cells on a Thermanox® square directly below the microthreads, which allowed for the smallest possible volume of cell suspension for optimal cell attachment. The current seeding method does not ensure all cells in the cell suspension will be exposed to the scaffold surface since the channel is higher and wider than the braided scaffold. This is supported by the observations that only 23-30% of the cells in the suspension attached to the braided collagen scaffolds.

Research by Cornwell et al. on the migration of fibroblast cells onto single uncrosslinked and EDC/NHS crosslinked microthreads showed a higher affinity for fibroblast migration onto uncrosslinked threads, but uncrosslinked braided collagen threads had significantly less attachment of MDFCs than all surface modifications. This difference could be explained by protocol differences, in which the single threads were EDC/NHS crosslinked in the presence of distilled water whereas the braided scaffolds were crosslinked in ethanol. Pieper et al. showed fibroblasts and myoblasts were more viable when collagen sponges were crosslinked in the presence of ethanol. In the teachings of this disclosure, heparin promoted significantly higher attachment of fibroblast cells, but other studies show the effect on attachment is not consistent with our findings. The attachment assay disclosed herein shows that varying the concentration of FGF-2 does not facilitate cell attachment. Similarly, Cornwell et al. loaded fibrin thread bundles with increasing concentrations of FGF-2, from 0 to 200 ng/mL, in the absence of heparin and observed that FGF-2 did not increase the attachment of fibroblast cells over the bundles without FGF-2. Interestingly, with the addition of FGF-2 to EDC/NHS with heparin scaffolds, the attachment between EDC/NHS crosslinked scaffolds and FGF-2 bound scaffolds were not significantly different. By binding FGF-2 to the heparin, the effects of the heparin on cell attachment are blocked leaving only the crosslinked surface to facilitate attachment.

The methods of these teachings showed that after five days, there was no significant increase in the number of MDFCs on the surface of the braid relative to day 1, except for scaffolds modified with EDC/NHS crosslinking and 5 ng/mL and 10 ng/mL FGF-2. After 7 days in culture, all braided collagen scaffolds with different modified surfaces, including uncrosslinked controls, promoted significant cell growth of MDFCs. A positive linear correlation was observed between the amount of FGF-2 on the surface of the scaffold and the rate of cell growth. The MDFCs grew uniformly across each surface type of the braids for the entirety of the culture period.

After 5 days in culture, the cell growth on each scaffold was insignificant showing no difference in scaffolds modified EDC/NHS with heparin and a 1.1 fold increase in scaffolds modified with EDC/NHS with heparin and 50 ng/mL FGF-2. The growth on modified surfaces was significantly higher than on uncrosslinked controls. Previous research supports the finding that the minimal growth of cells on scaffolds conjugated with heparin. It has been shown that heparin does not facilitate proliferation of endothelial cells on collagen films. Unexpectedly, scaffolds modified with 5 ng/mL FGF-2 showed significant increases in growth over the 1 day data as well as a significant increases in growth over scaffolds exposed to 10 ng/mL FGF-2.

The observed similarity between the growth on controls and FGF-2 modified surfaces when normalized to attachment is expected because when FGF-2 is bound to heparin binding sites it is released at a controlled rate compared to binding it to the collagen scaffold directly. Another reason for the limited growth after 5 days is that studies show that at low cell seeding densities, less than 10,000 cells/cm², FGF-2 does not begin to influence proliferation of endothelial cells until after 6 days in culture. In the study performed by Wissink et al., they used a collagen film that was over 2 cm² in surface area with a higher concentration of FGF-2 on the surface then on braided collagen scaffolds. The significantly smaller surface area with less FGF-2 per square centimeter explains why the cells in contact with FGF-2 on the braids had the same growth reaction even though there were more cells per square centimeter. Another explanation for insignificant growth in cells at 5 days on the braided collagen scaffolds could be due to the decreased proliferation. Due to collagen autofluorescence, the cells could not be evaluated for viability after attachment, so it is not known if there was rapid cell death during the first few days in culture.

After 7 days in culture, the cells attached to the scaffolds have begun to proliferate showing significant growth on all surface modifications. The different concentrations of FGF-2 on the surface of the braids appear to influence the growth rate of the MDFCs. Scaffolds modified using EDC/NHS crosslinking with heparin and exposed to 50 ng/mL FGF-2 increased 1.8 fold over the amount of cells initially attached to the surface, which was a significant increase over the growth after 5 days in culture. The growth rate of cells on braided collagen scaffolds were significantly higher than all control conditions, except there was no significant difference at day 7 between uncrosslinked and 5 ng/mL FGF-2 scaffolds.

Other studies have also observed similar effects of FGF-2 on the proliferation of cells on biodegradable scaffolds. To assess for the effect of FGF-2 on the proliferation of fibroblasts attached to fibrin microthreads, Cornwell et al. loaded varying concentrations of FGF-2 to bundles of fibrin microthreads and found after 2 days fibroblasts had growth 3 to 4 times over the attached number of cells, with a FGF-2 response plateau at 50 ng/mL. The difference in initial growth on fibrin microthreads compared to braided collagen scaffolds can be explained by the addition of heparin binding on the braids. When FGF-2 is bound to the scaffold directly by nonspecific interactions, there is an initial burst of the protein in the first 6 hours, which explains why the fibrin threads show a FGF-2 response after 2 days in culture. The high affinity of FGF-2 for heparin binding sites allows for the protein to be released much slower, with only 15% of the protein being released after 2 days compared to almost 60% when not bound to heparin. After 7 days in culture, fibroblast seeded to fibrin microthreads showed a similar growth pattern as on braided collagen scaffolds seeded with MDFCs with growth increasing with increased FGF-2 up to fibrin threads loaded with 50 ng/mL.

In other studies, collagen films and PLGA scaffolds crosslinked with EDC/NHS in the presence of heparin with increasing concentrations of FGF-2 showed a controlled release of FGF-2 from the scaffold when seeded with endothelial cells after 10 days. Scaffolds exposed to FGF-2 without heparin showed lower amounts of bound FGF-2 with unpredictable proliferation rates up to 10 days. Heparin scaffolds had increased amounts of FGF-2 on the surface with proliferation rate increasing in proportion to the concentration of FGF-2. FGF-2 binds to heparin molecules by electrostatically binding through interactions between the 2-O-sulfate groups and N-sulfate groups of the heparin binding sites with lysine and arginine residues on FGF-2 proteins. The effect FGF-2 has on the growth of the MDFCs on the braided collagen scaffolds suggests that the heparin has protected the protein from the enzymatic degradation mediated by MDFCs.

Hill et al. transplanted myoblasts seeded on alginate gels containing FGF-2 and HGF into mouse models and showed that the addition of growth factors increased transplanted cell participation in native muscle regeneration by promoting cell migration of both native and transplanted myoblasts. The study showed that it is imperative to determine the optimal rate of FGF-2 release to control myoblast viability and migration without initiating a myogenesis response in the surrounding tissue. Although the data herein indicates that the FGF-2 is bound to the collagen scaffolds by means of the heparin binding sites, the amount of heparin and FGF-2 on the surface has yet to be analyzed. It is envisioned that the surface characteristics of these scaffolds will be determined in order to decipher the optimal release rate of FGF-2. In addition, it is envisioned that the amount of FGF-2 needed to reach saturation of heparin binding sites and growth effects will be determined.

### Qualitative Analysis of Cell Density and Cellular Alignment

### Histological Analysis of Cell Density

To assess cell density and nuclei conformation on braided collagen thread scaffolds, cell outgrowth and alignment were evaluated after 1 or 7 days with each surface modification type as well as unseeded controls. Scaffolds were fixed in 4% paraformaldehyde solution in PBS for 40 minutes at room temperature, washed twice for 5 minutes in PBS. Prior to placement in tissue cassettes (Fisher Scientific, Pittsburgh, Pa.), samples were embedded in 2% (wt/vol) Lonza SeaKem LE Agarose (Fisher Scientific, Pittsburgh, Pa.) in distilled water to maintain the structural characteristics of the braid, and then all samples in tissue cassettes were placed into 70% (vol/vol) ethanol overnight. Next, scaffolds were processed for embedding by dehydrating in a series of increasing concentrations of ethanol, from 70% (vol/vol) to 100% (vol/vol), cleared with xylene, and embedded in paraffin wax at 60° C. Samples were embedded to analyze the cross section of the braid by cutting the braid orthogonal to the long axis and mounting the cut pieces vertically in the paraffin. Sections were cut at 5 μm on a rotary microtome (Nikon), mounted on Superfrost Plus slides (VWR, West Chester, Pa.) with Permount (Fisher Scientific, Pittsburgh, Pa.), and stained with Modified Harris Hematoxylin and Eosin (H&E; Richard-Allan Scientific, Kalamazoo, Mich.). Sections were imaged using an Olympus IX81 motorized inverted microscope coupled to a 12-bit Hamamatzu CCD camera and processed using Slidebook® to determine cell density.

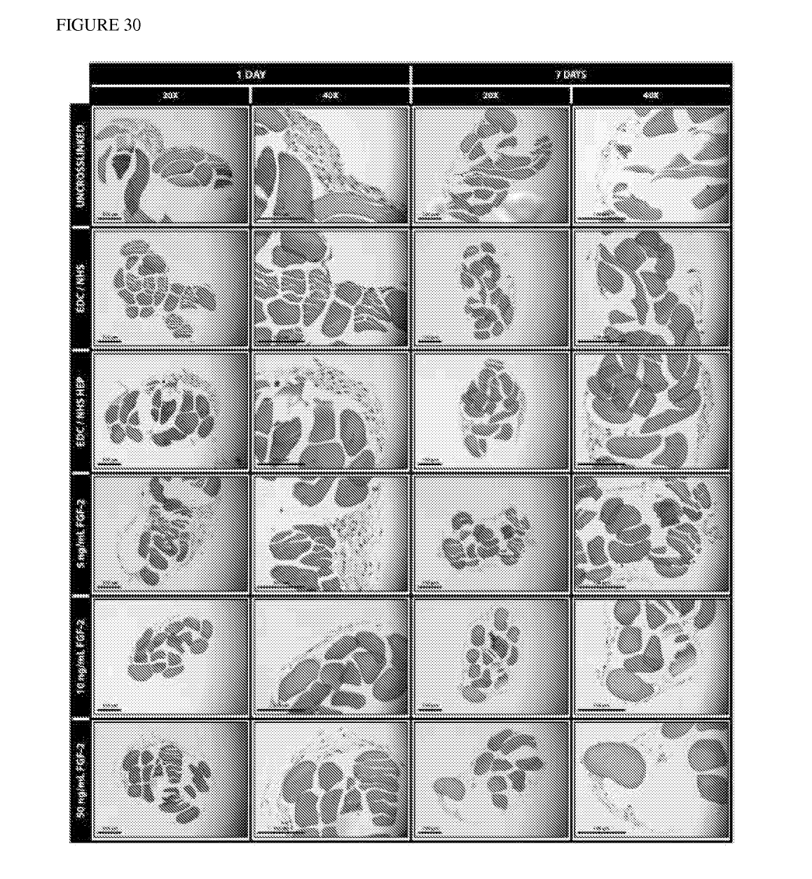

Differences in cell density on braided collagen scaffolds with different surface modifications was analyzed using histological sections of MDFCs on braids after 1 and 7 days in culture stained with H&E. The sections were imaged to determine qualitatively the cell thickness and homogeneity on the surface of the braid (FIG. 30). After 1 day in culture, the MDFCs are located on the surface of the braided collagen scaffolds showing very little spreading. The cells are attached to the braids in clusters with heterogeneous cell density distributions. After 7 days, the amount of cells on the surface seems to have decreased, but the cells appear more uniformly spread throughout the surface of the braid creating a more homogenous thickness.

### Fluorescence Microscopic Analysis of Cell Density and Cellular Alignment

To determine MDFC alignment and orientation on braided collagen scaffolds, scaffolds were seeded with MDFCs, incubated and stained to illuminate the f-actin filaments. Braided collagen scaffolds of each type were assembled and seeded as described previously and incubated for 1, 5, or 7 days. After incubation for the designated period, scaffolds were rinsed twice in PBS and fixed with 4% paraformaldehyde solution in PBS for 20 minutes at room temperature 88 phalloidin (Molecular Probes, Eugene, Oreg.) for 45 minutes. To image the braided scaffolds, they were removed from PDMS rings and placed on a glass slide covered with enough PBS to maintain hydration throughout the imaging process. To analyze cell density, scaffolds were imaged by fluorescence microscopy on an Olympus IX81 motorized inverted microscope coupled to a 12-bit Hamamatzu CCD camera and processed using Slidebook® under 4× magnification to visualize the Hoechst stained nuclei. Cellular alignment was determined by removing scaffolds incubated for 1 or 7 days from PDMS rings and placing them into 35 mm diameter glass bottom culture dishes with a 10 mm diameter cover slip in the middle with a thickness of 0.19 mm (MatTek Corporation, Ashland, Mass.). The braids were held flat against the cover glass surface using vacuum grease and covered with enough PBS to maintain hydration throughout the imaging process. The scaffolds were imaged using fluorescence microscopy on a Leica TCS SP5 II point scanning confocal microscope (Leica Microsystems Inc., Bannockburn, Ill.) under an oil immersion 20× magnification lens to visualize the nuclei and f-actin filaments. Images were taken along the z-axis at a depth of 100 to 150 μm of the braided collagen scaffold. Cellular alignment was qualitatively analyzed by determining if the cells aligned with the curvature of the braids or parallel to the x-axis after 7 days in culture.

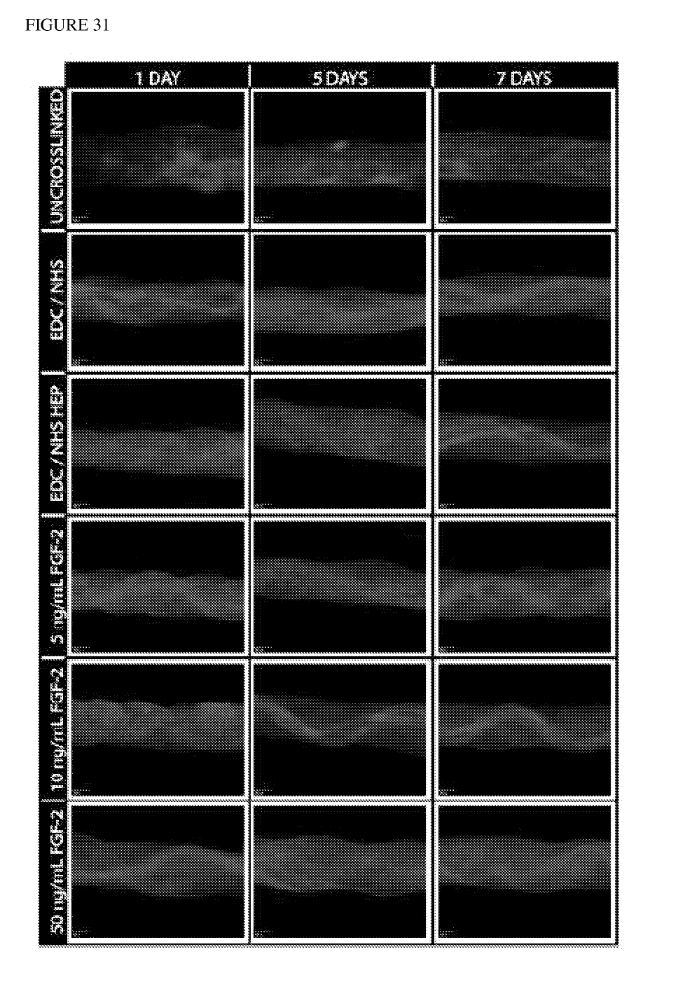

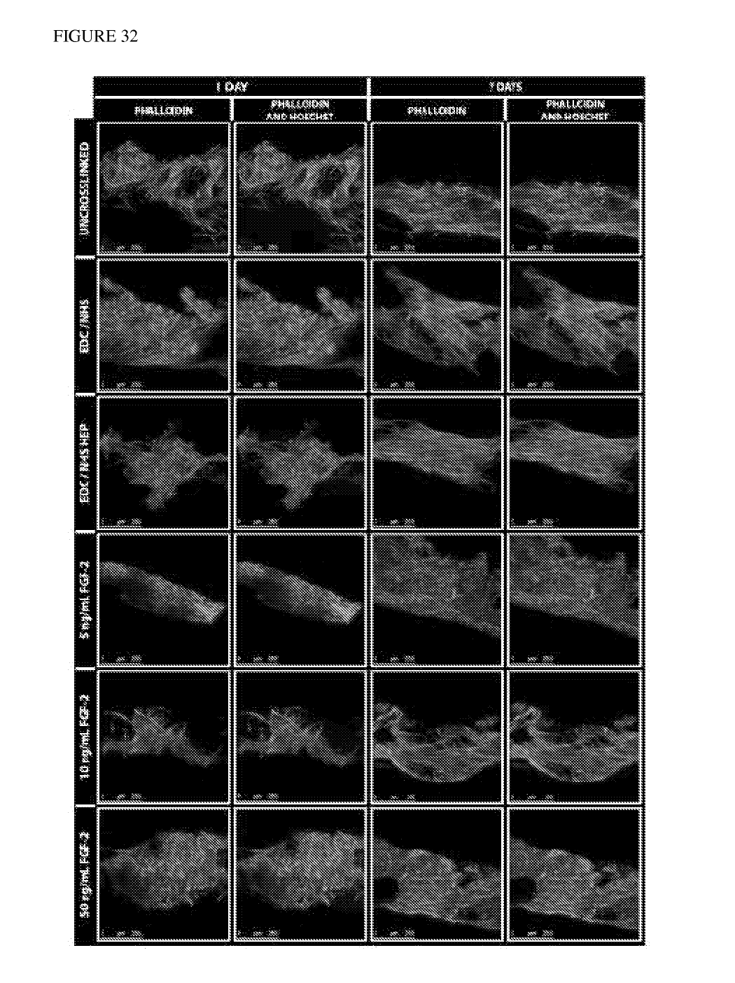

The effect of FGF-2 surface modifications on MDFC cell density and cellular alignment was determined by seeding MDFCs and incubating them on braided collagen scaffolds for 1, 5, and 7 days. The scaffolds were imaged either to determine cell density using Hoechst dye fluorescence microscopy, or to determine cellular alignment using phalloidin confocal fluorescence microscopy. The Hoechst stained cells at 1, 5, or 7 days shows uniform cell density over each surface modification (FIG. 31). The uncrosslinked scaffolds show a higher concentration of cells in the grooves between threads, and all braided scaffolds with surface modification showed a more uniform density across the entirety of the braid. The uniform concentration of cells indicates that imaging a small subsection of the braid would be a satisfactory representation of the alignment over the seeded area. Since the working distance of the confocal microscope was not large enough to image through the entire scaffold, only a small fraction of the braid could be analyzed per image. Confocal images of phalloidin stained braids at 1 day and 7 days, showed a distinct difference in the f-actin configurations between the two time points (FIG. 32). At 1 day, all scaffolds exhibit a lack of cellular alignment, with f-actin filaments spread out with no specific orientation. At 7 days, the cells began to orient themselves along the linear axis, meaning the direction of the threads not accounting for the curvature of the braids, on uncrosslinked braids and braids crosslinked with and without heparin. The f-actin filaments appear to be aligned parallel to each other over the braid structure with some following the curvature of the individual braided threads. Braided collagen scaffolds modified with different concentrations of FGF-2 showed limited alignment resembling the 1 day scaffolds as opposed to the 7 day uncrosslinked and crosslinked with and without heparin scaffolds.

In order to determine how the MDFCs are growing and spreading on the braided collagen scaffolds, the scaffolds were fixed, sectioned, and stained using H&E after 1 or 7 days in culture. The results showed that after 1 day, the cells were clustered on the surface of the side exposed during the seeding process. After 7 days, the cells are more spread along the surface, but the density and thickness is less than that at 1 day, which could be attributed to the cell spreading or limitations associated with fixing and sectioning the braided collagen scaffolds. Since, it was shown herein that the number of cells on the scaffolds increases with time on each surface, the apparent decrease in cell number at day 7 could be caused by cells shearing off the surface during processing or by sectioning artifacts. During sectioning, it appeared that some of the braids were not embedding vertically, which is supported by the observation that not all sections contained 18 individual thread cross sections. In addition, the braids did not maintain the tight structure in which they were cultured, so when sectioned, the threads spread, which detached the cells from the surface. This is observed by the cells seen in the cross sections that are not near any collagen threads, such as with the scaffolds modified with 50 ng/mL FGF-2 at 7 days in culture. The limitations of analyzing the braided collagen scaffolds thorough histology could be corrected by fixing the scaffolds more thoroughly or using a higher concentration of agarose to form a stiffer encapsulation around the braids. Using a 2% agarose in distilled water to maintain the structure may not be stiff enough to immobilize the scaffold during cutting.

An important aspect of engineered skeletal muscle constructs is how the scaffold facilitates alignment of the myotubes during regeneration. Myotubes, which are responsible for movement of the body, consist of bundles of linearly aligned myofibrils composed of fused myoblasts. The engineered skeletal muscle should stimulate the alignment and fusion of myoblasts to mimic the native environment, and allow for optimal integration and function upon implantation into a wound area. To analyze the alignment of MDFCs on braided collagen scaffolds with different surface modifications, scaffolds seeded with MDFCs were imaged using a confocal microscope after 1 or 7 days in culture. At one day, phalloidin staining of the f-actin filaments on all surface modifications showed no distinct orientation. After 7 days in culture, all control scaffolds have cellular alignment along the linear axis of the braided collagen microthreads, but scaffolds modified with FGF-2 show limited alignment with most f-actin filaments having no specific orientation.

It may be important to have the MDFCs aligned in the dedifferentiated state because it will facilitate the fusion of myoblasts with themselves and with host cells when the iPS cells are programmed to differentiate for muscle regeneration. By using microthreads, the myofiber-like structure of the biomaterial scaffold will promote the MDFCs to spread and proliferate along the linear axis of the braids. This will produce an organized skeletal muscle structure that will easily integrate into a large muscle defect. Braided collagen scaffolds modified using EDC/NHS crosslinking with heparin and FGF-2 showed limited alignment after 7 days, which is not consistent with previous research. Cornwell et al. observed bundles of fibrin microthreads with FGF-2 stimulated fibroblast cells alignment along the linear axis of the threads. One explanation of the limited alignment of MDFCs on FGF-2 modified braids could be the increased proliferation between 5 and 7 days in culture compared to the control scaffolds. With increased proliferation, the MDFCs could begin to stack on top of one another, limiting the contact of the MDFCs have with the braided collagen scaffold, which could eliminate the influence of the braid structure has on the alignment of the cells. It is envisioned that the MDFC orientation and alignment on the FGF-2 modified braided collagen scaffolds will be analyzed between 1 and 7 days in culture as well as in prolonged studies to determine when MDFCs begin to align. It is envisioned that the viability of the cells will be determined on the braided muscle construct as the cells proliferate and migrate outward to determine if there is a perfusion of nutrients to the inner layers, which can be an issue in three-dimensional engineered tissues. It is envisioned that alignment can be created by using magnetic, electrical, and mechanical stimulation. For example, exposing myogenic cells to a continuous magnetic field helps initiate the alignment, differentiation, and fusion of myoblasts into myotubes.

Studies have also shown that using aligned electrospun nanofibers seeded with myoblasts for muscle regeneration can promote the fusion of longer aligned myotubes after 7 days. After 14 days, cell nuclei begin to elongate and fuse exhibiting fast myosin heavy chains, which indicates the production of mature myotubes. Therefore, it is envisioned that braided collagen scaffolds, which supported the alignment of MDFCs along the longitudinal axis of the braids after 7 days, will create aligned engineered muscle once MDFCs are programmed to differentiate into terminal myoblasts.

Researchers have also utilized microfluidic patterning to create channels to promote myofiber alignment. Lam et al. investigated the effect of using different channel widths on myofiber alignment showing that widths of 6 μm promoted optimal alignment. By seeding myoblasts into synthetic polymer molds, the myoblasts are able to align parallel to the microgrooves, which then can be transferred into a collagen gel to create a three-dimensional muscle construct. Zhao et al. showed potential for creating multilayer muscle constructs using microfluidics, but when adding cell layers to the aligned myofibers in the channels, the newly formed myotubes do not exhibit the same highly oriented alignment. Shimuzi et al. created micropatterns using an abrasive substrate, which is used for biomaterial implants, and microchannels created with rougher surfaces encouraged a higher degree of alignment in myotubes. A limitation of using microfluidics to pattern aligned myofibers is the fibers do not fuse with one another until transferred out of the polymer. It is envisioned that this effect may be corrected by using microthreads to align the cells since all cells in the structure are in direct contact with one another.

### Statistics

Statistical analyses were executed using SigmaPlot 11.0 (Systat Software, Inc, Point Richmond, Calif.). For the mechanical data analysis, statistical differences between the uncrosslinked and crosslinked samples were evaluated using a Student's T-test or the Mann Whitney Rank Sum test for cases of unequal variance. For all cell-based assay experiements, statistical difference between scaffolds was analyzed using one-way analysis of variance (ANOVA) with Holm-Sidak post hoc testing. In cases where data failed the normality test an ANOVA on Ranks followed by a Dunn's post hoc test was used since the group sizes were unequal. Significance was established for p<0.05.

### Implanting Microthreads

Fibrin microthreads are approximately 55-65 μm in diameter, which is sufficient to promote longitudinal growth and alignment of cells by contact guidance. Additionally, fibrin microthreads can be manipulated to modulate mechanical strength and degradation dynamics by ultraviolet or chemically-induced cross-linking, and allow the seeding of cells in vitro, thus acting as a delivery vehicle for autologous cell implantation.

### Derivation and Characterization of Cells

Muscle tissue from a 34-year old male was obtained from tissue discarded during a muscle flap autograft procedure. Tissue was transported in ice cold Leibowitz L-15 medium (Mediatech) supplemented with bacitracin, neomycin, penicillin, streptomycin (all from EMD), and amphotericin B (Fungizone, Mediatech). Skeletal muscle was dissected from fat and tendon, minced into small pieces, and digested in IMDM (Mediatech) with 0.1% collagenase type II and 0.1% dispase (both from Worthington) at 37° C. with rotation at 8 rpm for 30 min. The tissue suspension was passed through a 100 μm cell strainer and the pass-through collected; washed 2× in DPBS and placed in DMEM/F12 (Mediatech) with 10% FCIII (Hyclone). The procedure was repeated twice. The final tissue remnants were digested at 37° C. in IMDM with 0.05% trypsin for 10 min and passed through a 100 μm cell strainer. All pass-through fractions were pooled and washed in DMEM/F12 with 10% FCIII and pelletted by centrifugation (700×g for 5 min). Cell pellets were resuspended in DMEM:F12 (60:40, v/v) supplemented with 10% FCIII with 4 ng/ml FGF2 (PeproTech) and seeded into 100 mm glass tissue culture plates and incubated at 37° C. in a humidified atmosphere consisting of 5% O₂ and 5% CO₂ (ELS culture). Alternatively, cell pellets were resuspended in DMEM:F12 (60:40, v/v) supplemented with 10% FCIII, seeded into 100 mm polystyrene tissue culture plates and incubated at 37° C. in a humidified atmosphere consisting 5% CO₂ in air (Standard culture). Cells were cryopreserved at passage 1 for use in subsequent experiments. Human embryonic stem cells (WA09) were grown as recommended by the supplier (WiCell).

### Immunocytochemistry Analysis

For immunocytochemistry (ICC), cells were seeded at 5,000-10,000 cells/well into 24-well plates (Nunc) with or without 12 mm round #2 glass coverslips. Cells were fixed with ice-cold methanol for 10 min, rinsed with PBS and stored at 4° C. until use. Primary antibodies and their concentration were: OCT4 (2.5 μg/ml, SantaCruz), SOX2 (2.0 μg/ml, Sigma), NANOG (2.5 Abcam), MYOD1 (2.5 μg/ml, Abcam), MYOGENIN (2.5 μg/ml Abcam), DESMIN (2.5 μg/ml, Abcam), PAX7 (Developmental Studies Hybridoma Bank 1:50), and NESTIN (2.5 μg/ml, Abcam). Isotype appropriate secondary antibodies labeled with Alexafluor-568 (Invitrogen) were used at 4 μg/ml. Nuclei were counterstained with 0.5 μg/ml Hoechst 33342 (EMD). Fluorescence was visualized using an Olympus IX81 inverted microscope with appropriate excitation and cubes (Semrock, Brightline®). Images were captured using a 12-bit CCD camera (Hamamatsu) and processed using Slidebook® (Olympus).

### Reverse Transcriptase Polymerase Chain Reaction (RT-PCR) Analysis

RNA was extracted from cell pellets using 1 ml of Trizol Reagent (Invitrogen) according to the manufacturer's protocol. Following precipitation, the RNA pellet was reconstituted in 20 of DEPC-treated H₂O, and 10U of RNAse-free DNAse (Stratagene) was added followed by 30 minute incubation at 37° C. DNAse was heat inactivated by incubation at 85° C. for 10 min. RNA concentration was quantified using a NanoDrop 4000 (Thermo Scientific), and 1 μg of total RNA was used for reverse transcription using the qScript cDNA Supermix kit (Quanta Biosciences) according to the manufacturer's protocol. One tenth of the final RT reaction volume was combined with 250 μM each, of forward and reverse primer (determined using NCBI's PrimerBlast and synthesized by Integrated DNA Technologies), 10 μl of 2× GoTaq® Green Master Mix (Promega), and molecular-grade water was added to 20 μl. PCR conditions were as follows: Initial denaturation for 4 minutes at 95° C.; 35 cycles of 30 seconds at 95° C., 30 seconds at annealing temperature, 30 seconds at 72° C.; with a final extension at 72° C. for 4 minutes. All primer annealing was carried out at 60° C. with the exception of SOX2 where annealing was performed at 55° C. The total resulting PCR product was resolved in a 2% TBE agarose gel containing 0.005% ethidium bromide, and visualized by exposure to 535 nm light from a halogen source on a Kodak Image Station MM4000 (Eastman Kodak).

TABLE 9
Primers used for genomic PCR and RT-PCR

| | | | Expected |
|---|---|---|---|
| | | | Amplicon |
| Gene/ | Forward Primer | Reverse Primer | Size |
| Primer | Sequence, 5′-3′ | Sequence, 5′-3′ | (bp) |
| Alu-Sx | GGCGCGGTGGCTCACG | TTTTTTGAGACGGAGTCTCGCTC | 282 |
| Desmin | GACCACGCGCACCAACGAGA | CCGCTCGGAAGGCAGCCAAA | 335 |
| hGAPDH | ACAGTCAGCCGCATCTTCTT | TGGAAGATGGTGATGGGATT | 288 |
| mGAPDH | TGTTCCTACCCCCAATGTGT | TGTGAGGGAGATGCTCAGTG | 396 |
| MyoD1 | GCCGGACAGGAGAGGGAGGG | GGCCGGAACTTTCTGCCGCT | 103 |
| Myogenin | CAGGCCCTGCTCAGCTCCCT | TCGCAAGGATGCCCGGCTTG | 309 |
| Nanog | TTTAATAACCTTGGCTGCCG | AACACTCGGTGAAATCAGGG | 398 |
| Nestin | GAGCTGGCAAGGCGACTGGG | CCAGCTGCTGCCGACCTTCC | 337 |
| Oct4 | AGTTTGTGCCAGGGTTTTTG | TGTGTCTATCTACTGTGTCCCAGG | 197 |
| Pax7 | AAGATTCTTTGCCGCTACCA | GCGGCTAATCGAACTCACTAA | 222 |
| Sox2 | ACACCAATCCCATCCACACT | CAAACTTCCTGCAAAGCTCC | 223 |

### Microthread Fabrication and Cell Seeding

Fibrin microthreads made from human fibrinogen (Sigma, F4753) and bovine thrombin (Sigma, T4648) were prepared as previously described. In some experiments, carbon spheres (8-12 μm diameter, Sigma) were mixed with the thrombin solution prior to microthread extrusion to enable tracking of microthread location in vivo. Microthreads were sterilized with ethylene oxide and stored at −20° C. until use. For cell seeding, threads were cut into 2 cm lengths and placed into 5 ml snap-cap polystyrene tubes. Approximately 2 ml of medium containing 8×10⁴ cells was added to each tube. The tubes were placed into a rotator (Barnstead) fixed at a mild) (15° incline in a cell culture incubator, and rotated at 8 rpm for 24-48 hours. This procedure ensured 360° seeding around the cylindrical surface of the microthread.

### Wound Creation and Microthread Cell Transplantation

All animal protocols were approved by the IACUC at Worcester Polytechnic Institute and the ACURO at the DoD US Army Medical Research and Materiel Command. Female nude SCID (Strain SHO, Charles River Laboratories) mice about 6-10 weeks of age were anesthetized and all surgical procedures were performed under a stereomicroscope. The skin flap and fascia covering the tibialis anterior was retracted and a partial thickness central skeletal muscle defect was created by resection of 30-70 mm³ (2-3 mm×3-4 mm×5-6 mm) of tissue. Fibrin microthreads were placed into the wound with sterile forceps and cut to size. On average, 6-10 microthreads were used to fill the entire defect. Microthreads were secured at the proximal and distal ends of the defect using sequential drops of fibrinogen and thrombin solution. The skin flap was replaced over the wound and secured using 8-0 coated vicryl absorbable suture (Ethicon). Animals were allowed to recover under a heat lamp.

### Histological Characterization of Wound Sites

At appropriate time-points the animals were sacrificed and the tibialis anterior muscle exposed and dissected longitudinally away from the tibia leaving the ends attached. The approximate wound margins were marked with a histology marking pen. The whole lower leg was placed in 3.7% formalin for 1 hour, the muscle detached from the bone and fixed for an additional 2 hours at room temperature. Tissue was rinsed in PBS and stored at 4° C. until paraffin embedding. Sections (6 μm) were cut with orientation such that the wound bed and all 4 borders could be visualized. H&E and Masson's Trichrome sections were prepared using standard procedures.

For detection of human cells by immunohistochemistry (IHC), antigen retrieval was conducted in a pressure cooker for 20 min using citrate based antigen retrieval solution (Vector Laboratories). Immunostaining was done using Sequenza slide staining chambers (ThermoFisher) with primary anti-human nuclei antibody at 1:250 dilution (Chemicon) and anti-PAX 7 antibodies at 1:250 dilution (Developmental Studies Hybridoma Bank). Detection was performed using Impress anti-mouse IgG horseradish peroxidase based detection with Impact DAB (Vector Laboratories). Slides were counterstained with eosin and mounted using Crystalmount (Electron Microscopy Sciences). Histology and IHC images were visualized on Olympus IX-81 Inverted microscope and acquired with a Q-color™ camera using Q-capture Pro™ software and bright field illumination.

### Collagen Quantification

Collagen content in the wound bed was calculated by image analysis of Trichrome stained histological images taken at 10× magnification. JPEG images were taken at serial sections through the wound bed at various locations and imported into MATLAB. Color separations were performed and an analysis threshold (minimum grey value for the blue channel) was established for each image series collected using the same brightness and white balance settings. Output images showing only the computed blue coverage were compared to the color images to ensure the representation of truly blue color due to collagen staining. This method enabled the exclusion of signal in the blue channel due to staining of other structures, which were below the analysis threshold value. As a control, the analysis was performed on uninjured (control) mouse skeletal muscle tissue sections stained with Trichrome and collected using the same camera and threshold settings to confirm a collagen content of zero for control tissue. The ratio of blue pixels above the threshold to total pixels in the image was used to calculate the collagen content for each image.

### Statistical Analyses

For experiments in which quantification was performed, a two-tailed T-test assuming unequal variance was carried-out, pair wise, on each group using Microsoft Excel. Only those groups displaying differences at a confidence level of 5% (P<0.05) were considered statistically significant.

### Characterization of Human Muscle Cells in Extended Lifespan System (ELS) Culture

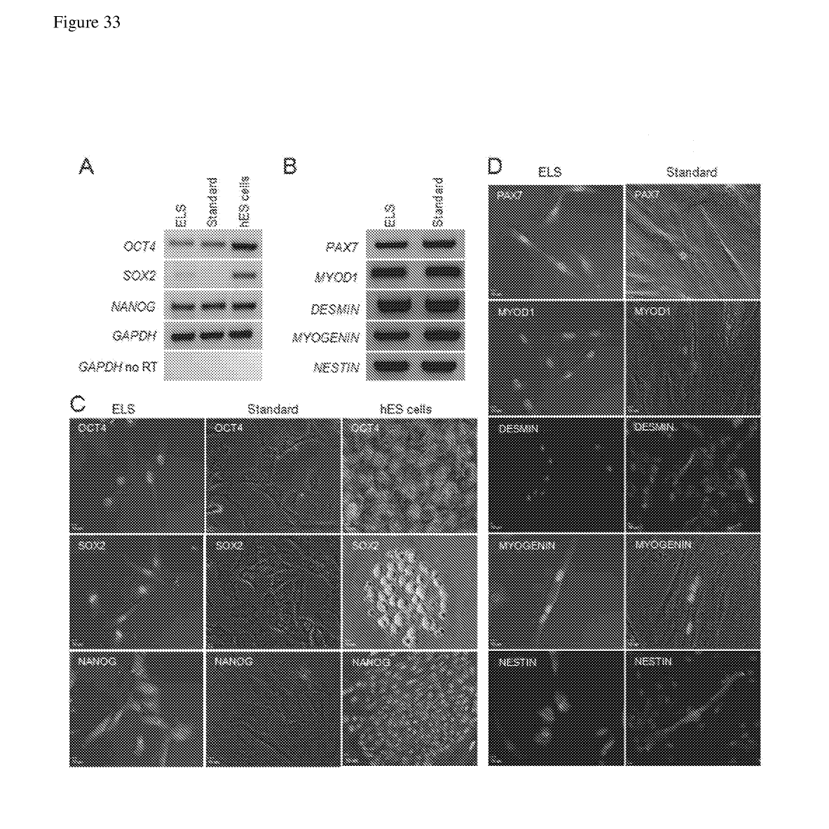

Expression of stem cell related transcripts (OCT4, SOX2, and NANOG, FIG. 33A) was detected in primary cells derived in both ELS and standard culture conditions, albeit at subjectively lower levels than in hESCs. Additionally, cells derived in both ELS and standard culture conditions also expressed transcripts for the myogenic genes PAX7, MYOD1, DESMIN, MYOGENIN, and NESTIN (FIG. 33B). Amplification of the housekeeping gene GAPDH at approximately equal intensities from all samples indicated equal input of cDNA, and the absence of a GAPDH amplicon from reactions without the addition of reverse transcriptase indicated the absence of any genomic DNA contamination.

Immunocytochemsitry (ICC) analysis detected the presence of all respective pluripotency-associated proteins in the nuclei of clusters of cells derived and cultured in ELS. The staining pattern was similar to that detected in hESCs used as positive controls, although the cell morphology remained fibroblastic (FIG. 33C). Cells cultured in ELS also displayed PAX7 staining and less DESMIN staining, although cells in both treatment groups stained positive for myogenic markers MYOD1, MYOGENIN and NESTIN (FIG. 33D). Cells cultured in the standard system lacked detectable OCT4, SOX2, NANOG (FIG. 33C), and PAX7 protein expression (FIG. 33D), clustered more in the center of the wells, and underwent spontaneous differentiation to form multi-nucleated myotubes (FIG. 33D). Cells derived and cultured in ELS and then transferred to standard conditions spontaneously differentiated into myotubes after FGF2 withdrawal, indicating that their myogenic potential was retained (data not shown). Taken together, these data demonstrate that the cell derivation technique can induce the translation of pluripotency-associated genes in primary human muscle-derived cells without disturbing myogenic cell fate and potential.

### Fibrin Microthreads Serve as an Efficient Delivery Vehicle for Human Muscle Progenitor Cells

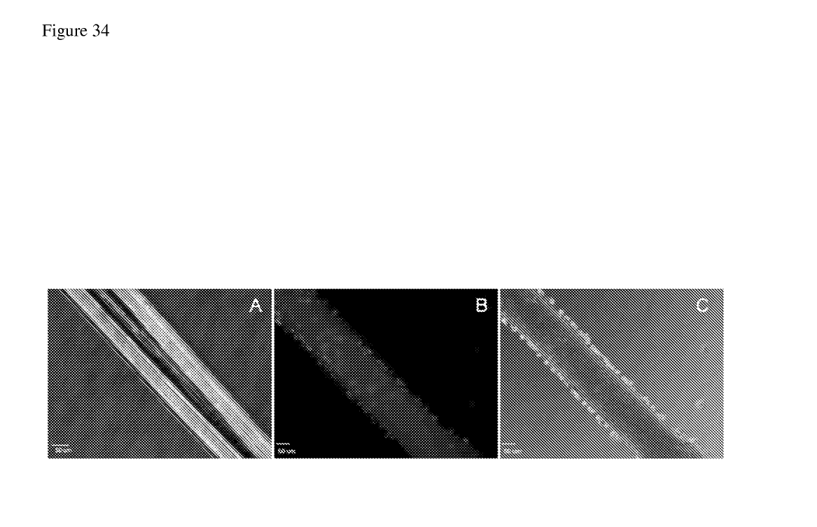

Cells were seeded onto fibrin microthreads (FIG. 34A) to assess the potential of microthreads as a delivery vehicle for muscle-derived cells, and to evaluate the efficacy of cells grown in ELS culture compared to conventional tissue culture methods, we seeded cells as described above. Cell seeding was confirmed by incubation with Hoechst to stain cell nuclei (FIG. 34B) and with phase contrast microscopy (FIG. 34C).

### Fibrin Microthreads Reduce Scar Formation and Promote Native Muscle Regeneration

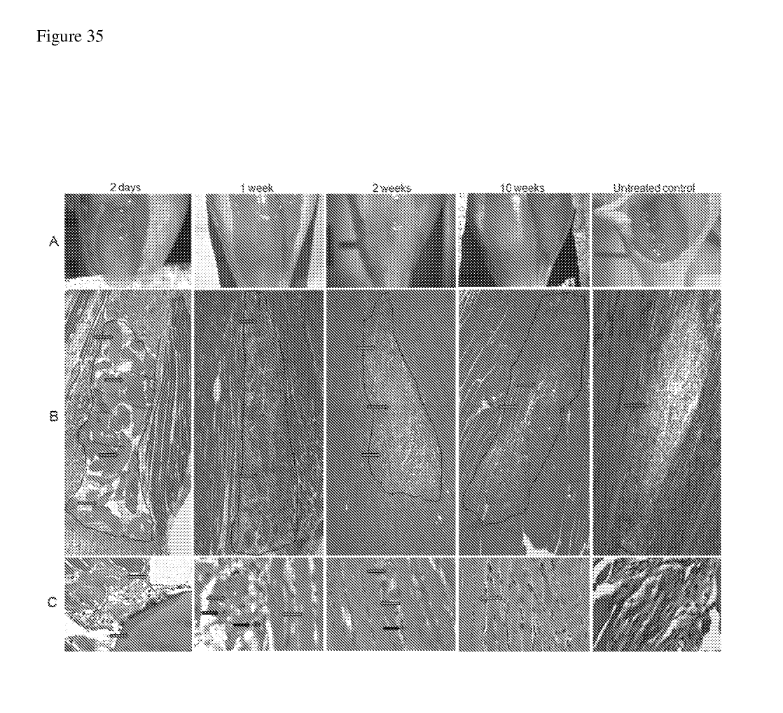

Replacing resected muscle tissue with fibrin microthreads was performed under a stereomicroscope. The position and size of the microthreads was customized at the time of surgery to the irregular shape of the defect by manually placing individual microthreads into the defect. Gross anatomical analysis at 2 days post surgery suggested that attachment of the microthreads to the host tissue was preserved (FIG. 35A). By 1 and 2 week time-points, microthreads were no longer individually visible by gross topical inspection (FIG. 35A). There was also evidence of physical contact between implanted fibrin and the host tissue at wound margins. Low magnification composite reconstructions of the entire wound site after Masson's Trichrome/eosin staining of sections at 2-day, 1-week, 2-week, and 10-week time-points illustrate the dynamics of the wound healing process (FIG. 35B). Microthreads were clearly visible at 2-day and 1-week time-points (red arrows) with evidence of their deterioration or degradation by a host response at 1-week. At 2 weeks, the wound site began to be reconstituted with new muscle fibers (grey arrows) in implanted wounds. In untreated control wounds, as expected, collagen (blue arrows) appeared to be the predominant constituent of the healed wound (FIG. 35B).

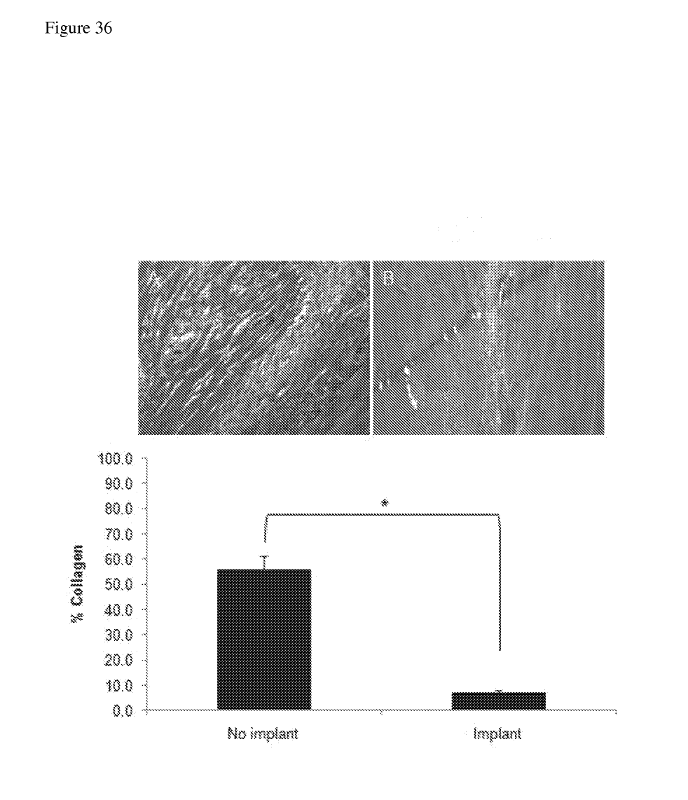

When imaged at higher magnification (FIG. 35C), areas where fibrin microthreads had been placed were noted by the presence of carbon particles extruded with the microthreads. At early time-points (1- and 2-weeks), carbon particles were visible near or within intact fibrin microthreads and adjacent to newly formed muscle fibers. The presence of newly regenerated muscle fibers was assessed based on the presence of multinucleated cells with centrally located nuclei. In many areas containing new muscle fibers, carbon particles were also visible. For later time-points, low magnification images of the entire wound bed revealed substantial new tissue in-growth in animals implanted with fibrin microthreads compared to large regions of mature collagen deposition in no-implant controls (FIG. 35C). Quantification of collagen deposition in treated and control wounds 30 days after surgery demonstrated that wounds that received an implant contained significantly less collagen than untreated controls (FIG. 36).

### Implanted Human Cells Participate in Mouse Muscle Healing Process

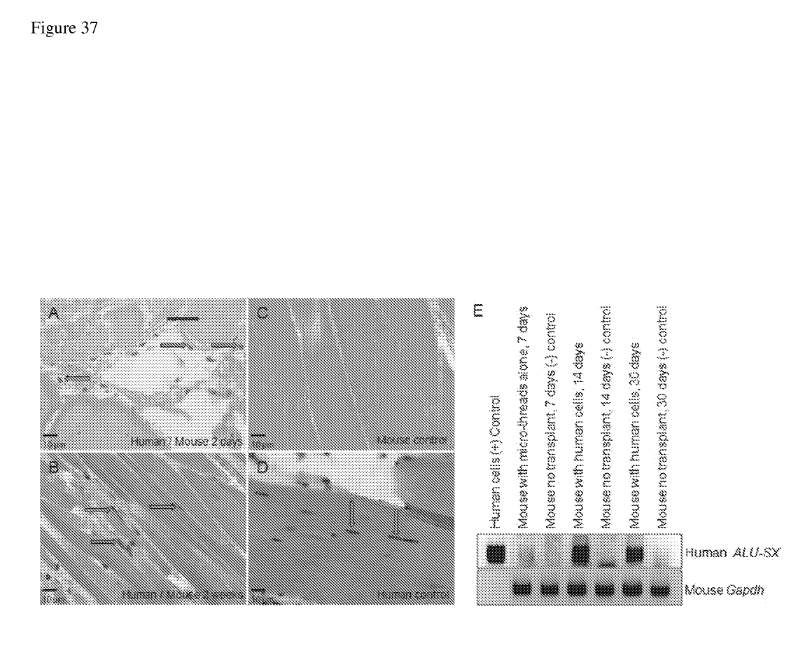

To evaluate the presence of implanted cells, the entire wound site was dissected at 2-days post implant and subjected to molecular analysis for the presence of human DNA by IHC and PCR. Evidence of human cell migration into the host stump tissue was detected at 2 days and 2 weeks post-implant by immunohistochemical (IHC) staining for human nuclear antigen (FIG. 37A). In addition, human nuclei were detected among apparently mature muscle fibers at 2-weeks by IHC (FIG. 37B). Human nuclei were detected in muscle fibers and in connective tissue. The specificity of the antibody was demonstrated by absence of labeling in control mouse muscle tissue and presence of labeling in control whole human muscle samples (FIGS. 37C and D, respectively). This was further confirmed by PCR analysis (FIG. 37E). Human DNA was detected in total DNA extracted from wound sites receiving human cell implants 30 days after surgery, but not from DNA extracted from non-implanted controls or controls implanted with microthreads alone (FIG. 37E).

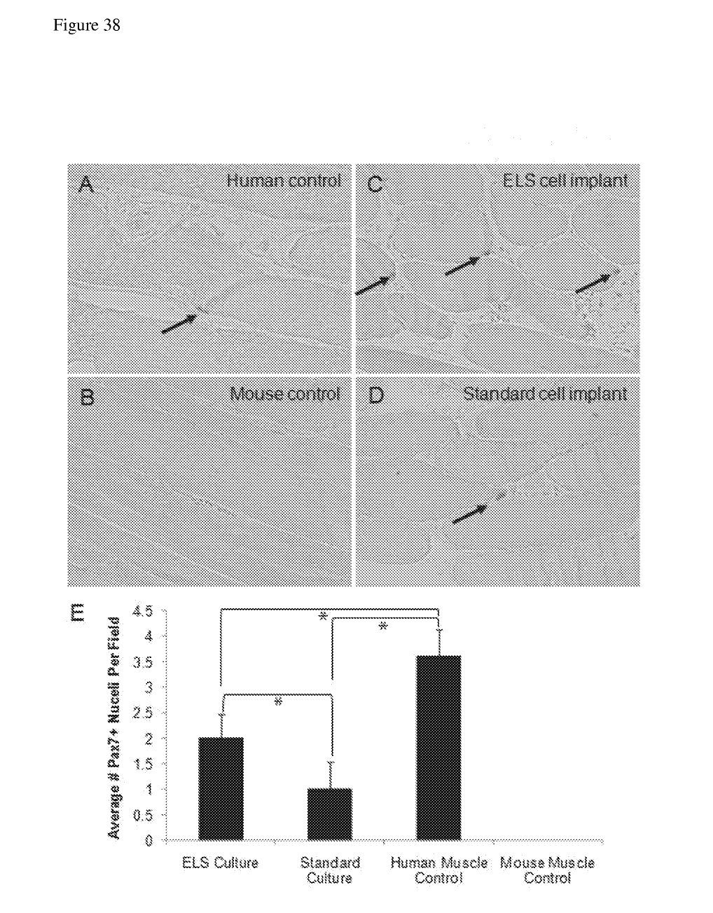

To determine if implanted cells were capable of contributing to the pool of host myogenic progenitor cells, sections of implanted tissue were stained by IHC with a human specific PAX7 antibody. Positive nuclei were detected in muscle tissue from human controls (FIG. 38A), mice implanted with cells cultured under ELS and standard culture conditions (FIGS. 38C and D, respectively), but not in non-implanted control mice (FIG. 38B). Wounds implanted with cells derived from ELS culture contained significantly more PAX7 positive cells than wounds implanted with cells derived in standard culture (FIG. 38E). These data indicate that our ELS culture is more capable of preserving myogenic progenitors than conventional culture systems, and this may be of relevance for skeletal muscle cell therapies as this technology moves toward the clinic.

The methods of these teachings demonstrate that fibrin microthreads can function as a suitable scaffold and cell therapy delivery vehicle for the repair of large skeletal muscle defects. Combined with fibrin glue anchorage at the wound margin, microthreads facilitate in-growth of nascent muscle tissue while reducing collagen deposition, as well as aid in engraftment of implanted progenitor cells previously cultured in vitro.

The ability to amplify tissue-specific cells in a less differentiated state in vitro prior to implantation improves regeneration by allowing for in vivo cues to direct differentiation, rather than setting the pathway in vitro using engineered skeletal muscle constructs prior to implantation. Although the mechanism is not understood at present, derivation and culture of primary skeletal muscle cells in physiological oxygen levels (5%) in conjunction with FGF2 supplementation, either preserves a more primitive cell phenotype and favors its propagation; or plays a role in the induction of a less differentiated phenotype. The latter possibility is supported by the observed expression of proteins associated with the pluripotent cell phenotype (OCT4, SOX2, and NANOG). Of specific note, high levels of FGF2 have been shown to support the maintenance of pluripotent human ES cells in feeder cell and serum free cultures, as well as supporting regeneration of a variety of tissues including skeletal muscle by affecting myogenic cells and angiogenesis. It was previously demonstrated that FGF2 supplementation in combination with tissue “normoxia” conditions (5% O₂), referred to herein as “ELS Culture”, extended the in vitro lifespan of primary human dermal fibroblast in a synergistic manner, and activated the expression of pluripotency-associated proteins. This culture system did not necessarily affect transcription of these genes, but rather protein translation, and even then, only in subpopulations of cells. Additionally, this phenomenon was dependent on the use of glass culture surfaces. Glass surfaces may render FGF2 more available to the cells through a direct property of the glass, or cell culture surface may alter extracellular matrix production, thereby modulating FGF2 availability.

A more primitive cellular phenotype can be achieved using the ELS culture system, and this is preserved following implantation into a muscle defect. Transplant of cells in a less differentiated state may further allow for new muscle fiber formation to occur concomitant with angiogenesis and/or innervation, rather than requiring these processes to be completed in pre-formed tissue. Whereas skeletal muscle satellite cells derived from mice have been shown to spontaneously enter altered non-myogenic phenotype following in vitro culture, and whereas substratum elasticity has been strongly implicated as a factor that controls satellite cell fate in vitro, our ELS culture system combining low oxygen and FGF2 supplementation on glass culture surfaces can address these issues and preserve a myogenic progenitor population. This is supported by the finding that animals receiving microthread implants containing cells cultured in the ELS system contain approximately twice the numbers of PAX7 positive cells as those animals receiving microthread implants seeded with cells cultured with standard techniques at 10 weeks following implantation, in addition to the detection of numerous mature human myotubes in implanted animals.

Fibrin microthreads alone contribute to wound healing. In animals receiving microthreads seeded with cells, although mature human muscle fibers were detected, the majority of newly reconstituted tissue was of host animal origin. Therefore, fibrin microthreads alone have tremendous potential for reducing fibrosis and remodeling large muscle injuries. A scaffold that approximates the target tissue architecture in terms of facilitating cell alignment offers an advantage over simple hydrogels which facilitate cell survival and engraftment. For treatment of large wounds, fibrin microthreads can both stabilize the wound and facilitate remodeling of the wound bed more completely than would be possible for full thickness hydrogels, while delivering progenitor cells anchored to a directional substrate into these deeper areas. The use of microthreads could alleviate the need to accomplish pre-vascularization to promote cell survival and preservation of cell phenotype inside fibrin hydrogels, as has been developed previously.

It is envisioned that microthreads will be used not only as a cell delivery vehicle, but also as a means to deliver pro-regenerative growth factors (such as FGF2) to an injury site. The incorporation of FGF2 into fibrin microthreads has been shown to enhance cell migration and proliferation of dermal fibroblasts in vitro. Additionally, controlled release of FGF2 from fibrin by manipulating both the fibrinogen concentration and the addition of heparin, which binds FGF2 and other growth factors, has also been demonstrated. The stability and kinetics of growth factor release from fibrin microthreads can be optimized to facilitate muscle defect healing by the methods of these teachings.

Fibrin microthreads in combination with an in vitro cell expansion system is disclosed herein for developing autologous cell therapies and scaffolds for the treatment of large skeletal muscle defects.

1. A composition comprising a polymer configured as a plurality of microthreads, each having a leading end and a trailing end for use as a therapeutic agent or device for repairing muscle tissue defects.

2. The composition of claim 1, further comprising a plurality of biological cells in association with the microthreads, and, optionally, a therapeutic agent.

3. The composition of claim 1, further comprising a surface modification of the plurality of microthreads.

4. The composition of claim 1, wherein the polymer configured as a plurality of microthreads comprises a naturally occurring polymer.

5. The composition of claim 4, wherein the naturally occurring polymer is collagen or fibrin.

6. The composition of claim 1, wherein the microthreads are braided, bundled or tied to form filaments.

7. The composition of claim 1, wherein the microthreads have a diameter of about 0.2-100 μm.

8. The composition of claim 2, wherein the cells are differentiated cells selected from the group consisting of: muscle-derived cells, muscle-derived fibroblastic cells, skeletal muscle satellite cells, satellite like cells, primary skeletal muscle cells, fibroblasts, and endothelial cells.

9. The composition of claim 8, wherein the cells is are muscle-derived fibroblastic cells.

10. The composition of claim 2, wherein the cells are undifferentiated or dedifferentiated cells selected from the group consisting of: dedifferentiated fibroblast cells, stem-like cells, myoblasts, induced pluripotent stem cells (iPS cells), muscle progenitor cells, embryonic stem cells, and mesenchymal stem cells.

11. The composition of claim 2, wherein the cells are autologous cells or allogeneic cells.

12. The composition of claim 2, wherein the therapeutic agent is a growth factor.

13. The composition of claim 12, wherein the growth factor is FGF-2.

14. The composition of claim 3, wherein the surface modification is crosslinking.

15. The composition of claim 3, wherein the surface modification is crosslinking with heparin.

16. A method of preparing a cell-containing composition for delivery to a patient, the method comprising culturing the composition of claim 1 with biological cells, wherein the cells become associated with the plurality of threads to form the cell-containing composition.
